(19) United States
(12) Patent Application Publication
(10) Pub. No.: US 2026/0124221 A1
(43) Pub. Date: May 7, 2026

(54) INJECTABLE PHARMACEUTICAL COMPOSITION FOR TREATMENT OF BREAST CANCER

(71) Applicant: KASHIV BIOSCIENCES, LLC, Piscataway, NJ (US)

(72) Inventors: Parva Yogeshchandra PUROHIT, Ahmedabad (IN); Ganesh Vishwanath SANGLE, Dist - Buldhana (IN); Sandip Pareshbhai MEHTA, Ahmedabad (IN); Vishal Bharatbhai UNADKAT, Ahmedabad (IN); Heta Nishil PANDYA, Ahmedabad (IN); Sagar Laxmanbhai VEKARIYA, Rajkot (IN)

(21) Appl. No.: 18/705,711

(22) PCT Filed: Oct. 29, 2022
(86) PCT No.: PCT/IB2022/060430

§ 371 Date: Apr. 29, 2024

(30) Foreign Application Priority Data
Oct. 29, 2021 (IN) 202121049524

(51) Int. Cl. A61K 31/661; A61K 9/00; A61K 47/10; A61K 47/14; A61K 47/18; A61K 47/22; A61K 47/34; A61K 47/44

(52) U.S. Cl. CPC A61K 31/661; A61K 9/0019; A61K 47/10; A61K 47/14; A61K 47/18; A61K 47/22; A61K 47/34; A61K 47/44

(57) ABSTRACT

The invention relates to an injectable composition comprising compound I, pharmaceutically acceptable salts or solvates thereof; and at least one pharmaceutically acceptable excipient, wherein the injectable composition provides sustained release of compound I and thereby maintains blood plasma fulvestrant concentration. Also, the present invention used for method of treatment of breast cancer comprising compound of formula I, pharmaceutically acceptable salts or solvates thereof; and at least one pharmaceutically acceptable excipient.

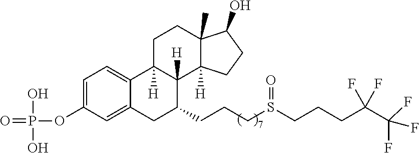

### FIELD OF INVENTION

The invention relates to an injectable composition comprising compound I,

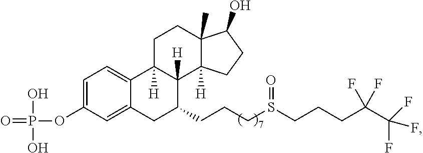

pharmaceutically acceptable salts or solvates thereof; and at least one pharmaceutically acceptable excipient wherein the injectable composition provides sustained release of compound I and thereby maintains blood plasma concentration of fulvestrant. The composition of the present invention is intended for intravenous or intramuscular administration by injection containing the compound I, pharmaceutically acceptable salts or solvates thereof; and at least one pharmaceutically acceptable excipient.

### BACKGROUND OF THE INVENTION

Well known estrogen receptor antagonist Fulvestrant is used for the treatment of hormone receptor positive metastatic breast cancer in postmenopausal women with disease progression following anti-estrogen therapy. Fulvestrant has clinical therapeutic effect in a subjects failed in treatment with tamoxifen which has initiated a new way of treating hormone-sensitive breast cancer.

Oestrogen deprivation is fundamental to the treatment of many benign and malignant diseases of the breast and reproductive tract. In premenopausal women, this is achieved by the ablation of ovarian function through surgical, radiotherapeutic, or medical means, and, in postmenopausal women, by the use of aromatase inhibitors.

Fulvestrant shows, along with other steroidal based compounds, certain physical properties which make composition of these compound difficult. Fulvestrant is highly lipophilic molecule, even when compared with other steroidal compounds, and its aqueous solubility is extremely low.

To maintains blood plasma Fulvestrant concentration, the release rate the amount of fulvestrant needed would require the composition volume to be large, at least 10 ml. This requires the doctor to inject an excessively large volume of fulvestrant composition significantly high for human therapy. A drug with poor solubility will often exhibit poor bioavailability and require administration of high dosages to attain therapeutically effective blood levels of the drug. Due to the poor solubility and oral bioavailability of fulvestrant, the drug is currently administered via intramuscular injection of an oil based Fulvestrant composition.

The Faslodex™ product is approved for administration by intramuscular injection on days 1, 15, 29, and once monthly thereafter. This injection contains castor oil which can be viscous and can be painful at the time of injection.

### SUMMARY OF THE INVENTION

The invention relates to an injectable composition comprising the compound I

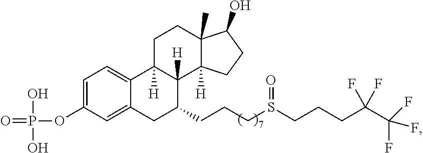

pharmaceutically acceptable salts or solvates thereof; and at least one pharmaceutically acceptable excipient, wherein the injectable composition provides sustained release of compound I and thereby maintains blood plasma concentration of fulvestrant for a desired period of time.

In one embodiment, the present invention provides an injectable composition containing the compound I, pharmaceutically acceptable salts or solvates thereof; and at least one pharmaceutically acceptable excipient, wherein an injectable sustained release composition is intended for intravenous or intramuscular administration.

In one embodiment, the invention relates to an intramuscular composition comprising the compound I, pharmaceutically acceptable salts or solvates thereof; and at least one pharmaceutically acceptable excipient, wherein the injectable composition provides sustained release of compound I and thereby maintains blood plasma concentration of fulvestrant from about 1 ng/ml to about 100 ng/ml for a desired period of time.

In one embodiment, the present invention provides a method for treating a hormonal dependent benign or malignant disease of the breast or reproductive tract comprising administering intramuscularly to a subject in need of such treatment the compound I, pharmaceutically acceptable salts or solvates thereof; and at least one pharmaceutically acceptable excipient.

In one embodiment, wherein the hormonal dependent benign or malignant disease of the breast or reproductive tract is breast cancer.

### BRIEF DESCRIPTION OF THE DRAWINGS

FIG. 1.—Mean±SD plasma concentration—time profile of fulvestrant following a single intramuscular administration of composition having compound I to female Sprague Dawley (SD) Rats.

FIG. 2.—Mean±SD plasma concentration—time profile of Compound I following a single intramuscular administration to female Sprague Dawley (SD) Rats.

FIG. 3.—Mean±SD plasma concentration—time profile of fulvestrant following a single intramuscular administration of composition having compound I to Female Beagle dogs.

FIG. 4.—Mean±SD plasma concentration—time profile of Compound I following a single intramuscular administration to Female Beagle dogs.

FIG. 5.—Mean±SD plasma concentration—time profile of Fulvestrant following a single intramuscular administration of composition having compound I to Female Beagle dogs.

FIG. 6.—Mean±SD plasma concentration—time profile of Compound I following a single intramuscular administration to Female Beagle dogs.

FIG. 7.—Mean±SD plasma concentration—time profile of Fulvestrant and compound I following a single intramuscular administration of Faslodex or composition having compound I to male cynomolgus monkeys.

### DETAILED DESCRIPTION OF THE INVENTION

Commercially available fulvestrant composition would require high volume for injection in order to achieve desired blood plasma fulvestrant concentration. This requires the physician to inject an excessively large volume of composition to administer an enough dose for human therapy. Hence, it is unmet need to have concentrated dosage form with low volume of injection and maintains blood plasma concentration of fulvestrant. Also, there is a significant unmet need exists for higher fulvestrant plasma concentrations to achieve better efficacy in the ESR1 mutant population; or to downsize the tumour which can decrease the likelihood of mastectomy.

Thus, the present invention provides an injectable composition comprising compound I, pharmaceutically acceptable salts or solvates thereof; and at least one pharmaceutically acceptable excipient which can provide low volume injection that maintains blood plasma fulvestrant concentration for desired period of time.

As used herein the term “intramuscular composition” refers to the composition intended for injecting into any intramuscular region of a subject in need thereof and thereby release the drug from the site of injection continuously. The site for intramuscular injection is selected from the arm (deltoid muscle), thigh muscles (vastus lateralis), the buttock (gluteal muscles).

As used herein the term “maintains blood plasma fulvestrant concentration” or “maintains blood plasma concentration of fulvestrant” refers to blood plasma concentrations of fulvestrant achieved from about 1 ng/ml to about 100 ng/ml in a subject for a desired period of time after administration of injectable composition having compound I, pharmaceutically acceptable salts or solvates thereof; and at least one pharmaceutically acceptable excipient.

As used herein the term “about” refers to +5% deviation from the said value.

As used herein, the term “salt” refers to an acid or base salt of a compound of the invention. Salts of basic compounds are salts formed with mineral acids, organic carboxylic acids, organic sulfonic acids, and the like. Examples of suitable acids include hydrochloric, hydrobromic, sulfuric, nitric, perchloric, fumaric, maleic, phosphoric, glycollic, lactic, salicylic, succinic, toluene-p-sulfonic, tartaric, acetic, citric, methanesulfonic, formic, benzoic, malonic, naphthalene-2-sulfonic and benzenesulfonic acids. Salts of acidic compounds are formed with bases, namely cationic species such as alkali and alkaline earth metal cations e.g., sodium, lithium, potassium, calcium, and magnesium ions as well as ammonium cations e.g., ammonium, trimethylammonium and diethylammonium. Salts of acidic compounds are formed with organic bases, namely tromethamine(tris) and meglumine. In the present invention, the salt formed with the compound I is selected from monovalent or divalent, for e.g. monosodium or disodium salt.

As used herein the term “sustained release” refers to a continuous release of the drug from the composition for a desired time period. Unless otherwise specified by any limitation in embodiment the term “sustained release” includes sustained release of compound I or fulvestrant after injecting the composition having compound I or, pharmaceutically acceptable salts or solvates thereof; and at least one pharmaceutically acceptable excipient.

As used herein the term “a subject” refers to animal or human. The animal is selected from rodent, non-rodent or mammals. The humans include healthy or diseased patient.

As used herein the term “standard fulvestrant injectable preparation” refers to Faslodex® or any Faslodex® equivalent composition of fulvestrant. It also refers to preparation that is either commercially available or prepared.

In one embodiment, the invention provides an injectable pharmaceutical composition adapted for administration by injection containing the compound I, pharmaceutically acceptable salts or solvates thereof; and at least one pharmaceutically acceptable excipient. More particularly composition intended for intravenous or intramuscular administration by injection containing the compound I, pharmaceutically acceptable salts or solvates thereof; and at least one pharmaceutically acceptable excipient.

In one embodiment, the pharmaceutical composition intended for the intravenous or intramuscular administration is selected from the dosage form of solution, emulsion, suspension, powders for injection or infusion, or gels for injection and implants. These are sterile preparations intended to be administrated directly into the human or animal body.

In one embodiment the present invention provides, a sustained release pharmaceutical composition comprising compound I, pharmaceutically acceptable salts or solvates thereof; and at least one pharmaceutically acceptable excipient intended for intramuscular and intravenous administration that maintains blood plasma concentration of fulvestrant after injecting the composition comprising compound I, pharmaceutically acceptable salts or solvates thereof. In a one embodiment, compound I, pharmaceutically acceptable salts or solvates thereof; in any compositions of the invention is present at concentration selected from about 1 to about 30% w/v, about 1-2% w/v, about 2-3% w/v, about 3-4% w/v, about 4-5% w/v, about 5-6% w/v, about 6-7% w/v, about 7-8% w/v, about 8-9% w/v, about 9-10% w/v, about 10-11% w/v, about 11-12% w/v, about 12-13% w/v, about 13-14% w/v, about 14-15% w/v, about 15-16% w/v, about 16-17% w/v, about 17-18% w/v, about 18-19% w/v, about 19-20% w/v, about 20-21% w/v, about 21-22% w/v, about 22-23% w/v, about 23-24% w/v, about 24-25% w/v, about 25-26% w/v, about 25-26% w/v, about 26-27% w/v, about 27-28% w/v, about 28-29% w/v or about 29-30% w/v. In a one embodiment, compound I, pharmaceutically acceptable salts or solvates thereof; in any compositions of the invention is present at concentration selected from about 1 to about 30% w/v, about 2 to about 30% w/v, about 3 to about 30% w/v, about 4 to about 30% w/v, about 5 to about 30% w/v or about 10 to about 30% w/v.

In one embodiment, the present invention provides the intramuscular composition comprising compound I,

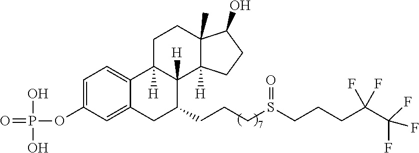

pharmaceutically acceptable salts or solvates thereof; and at least one pharmaceutically acceptable excipient, wherein the composition administered to a subject in need thereof comprises about 10 mg to about 1000 mg of compound I, pharmaceutically acceptable salts or solvates thereof. In one embodiment, the present invention provides the intramuscular composition comprising compound I, pharmaceutically acceptable salts or solvates thereof; and at least one pharmaceutically acceptable excipient, wherein the composition administered to a subject in need thereof comprises compound I, pharmaceutically acceptable salts or solvates thereof selected from group consisting of 50 mg to about 1000 mg, 100 mg to about 1000 mg, 150 mg to about 1000 mg, 50 mg to about 1000 mg, 100 mg to about 800 mg, 150 mg to about 800 mg, 50 mg to about 600 mg, 100 mg to about 600 mg, 150 mg to about 600 mg, 50 mg to about 500 mg, 100 mg to about 500 mg, 150 mg to about 500 mg.

In one embodiment, the present invention provides the intramuscular composition comprising compound I, pharmaceutically acceptable salts or solvates thereof; and at least one pharmaceutically acceptable excipient, wherein the composition administered to a subject in need thereof comprises compound I, pharmaceutically acceptable salts or solvates thereof selected from group consisting of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg.

In one embodiment, the present invention provides a method for treating a hormonal dependent benign or malignant disease of the breast or reproductive tract comprising administering intramuscular composition comprising compound I, pharmaceutically acceptable salts or solvates thereof; and at least one pharmaceutically acceptable excipient. In one embodiment, the method of treating a hormonal dependent benign or malignant disease of the breast is breast cancer.

In one embodiment, the present invention provides an injectable composition of compound I

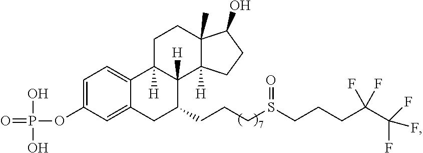

pharmaceutically acceptable salts or solvates thereof; and at least one pharmaceutically acceptable excipient, wherein the injectable composition provides sustained release of compound I and thereby maintains blood plasma concentration of fulvestrant. In one embodiment, the injectable composition can be intravenous composition or intramuscular composition. In one embodiment, the intramuscular composition comprising compound I, pharmaceutically acceptable salts or solvates thereof; and at least one pharmaceutically acceptable excipient which is intended to give sustained release of compound I in blood plasma and thereby maintains blood plasma fulvestrant concentration for a desired period of time.

In one embodiment, the intramuscular composition comprising compound I, pharmaceutically acceptable salts or solvates thereof; and at least one pharmaceutically acceptable excipient that maintains blood plasma fulvestrant concentration for at least one week after injecting the composition comprising compound I, pharmaceutically acceptable salts or solvates thereof.

In another embodiment, maintenance of blood plasma fulvestrant concentration is achieved for the desired period of time, which is selected from group consisting of one week, two weeks, three weeks, four weeks, one month, two months or three months after injecting the composition comprising compound I, pharmaceutically acceptable salts or solvates thereof.

In one embodiment, the present invention provides the injectable composition comprising a compound I, pharmaceutically acceptable salts or solvates thereof; and at least one pharmaceutically acceptable excipient, wherein the composition is administered intravenously to a subject in need of such treatment.

In one embodiment, the present invention provides the intramuscular composition comprising a compound I, pharmaceutically acceptable salts or solvates thereof; and at least one pharmaceutically acceptable excipient, wherein at least one pharmaceutically acceptable excipient is selected from oils, solvents, surfactants, antioxidant, polymer or a mixture thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml. In another embodiment, the composition of the present invention has the concentration of compound I, pharmaceutically acceptable salts or solvates thereof; and at least one pharmaceutically acceptable excipient, wherein the composition administered to a subject in need thereof has concentration of compound I, its pharmaceutically acceptable salts or solvates thereof is selected from about 20 mg/ml to about 300 mg/ml, about 30 mg/ml to about 300 mg/ml, about 40 mg/ml to about 300 mg/ml, about 50 mg/ml to about 300 mg/ml, about 60 mg/ml to about 300 mg/ml, about 70 mg/ml to about 300 mg/ml, about 80 mg/ml to about 300 mg/ml, about 90 mg/ml to about 300 mg/ml, about 100 mg/ml to about 300 mg/ml, about 110 mg/ml to about 300 mg/ml, about 120 mg/ml to about 300 mg/ml, about 130 mg/ml to about 300 mg/ml, about 140 mg/ml to about 300 mg/ml, about 150 mg/ml to about 300 mg/ml, about 160 mg/ml to about 300 mg/ml, about 170 mg/ml to about 300 mg/ml, about 180 mg/ml to about 300 mg/ml, about 190 mg/ml to about 300 mg/ml, about 200 mg/ml to about 300 mg/ml, about 210 mg/ml to about 300 mg/ml, about 220 mg/ml to about 300 mg/ml, about 230 mg/ml to about 300 mg/ml, about 240 mg/ml to about 300 mg/ml, about 250 mg/ml to about 300 mg/ml, about 260 mg/ml to about 300 mg/ml, about 270 mg/ml to about 300 mg/ml, about 280 mg/ml to about 300 mg/ml, or about 290 mg/ml to about 300 mg/ml.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof about 10 mg/ml. In one embodiment, the intramuscular composition comprising compound I or pharmaceutically acceptable salts and solvates thereof; and at least one pharmaceutically acceptable excipient, wherein the composition administered to a subject in need thereof has concentration of compound I or pharmaceutically acceptable salts or solvates thereof is selected from about 20 mg/ml, about 30 mg/ml, about 40 mg/ml, about 50 mg/ml, about 60 mg/ml, about 70, mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml, about 260 mg/ml, about 270 mg/ml, about 280 mg/ml, about 290 mg/ml, or about 300 mg/ml.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration for a desired period of time after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration for at least one week after injecting the composition comprising compound I, pharmaceutically acceptable salts or solvates thereof. In another embodiment, the composition comprising compound I, pharmaceutically acceptable salts or solvates thereof; and at least one pharmaceutically acceptable excipient, maintains blood plasma concentration of fulvestrant for the time selected from at least two weeks, at least three weeks, at least four weeks, at least one month, at least two months or at least three months after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of at least 1 ng/ml for desired period of time after injecting the composition comprising compound I, pharmaceutically acceptable salts or solvates thereof. In one embodiment, the intramuscular composition maintains blood plasma fulvestrant concentration of at least 1 ng/ml for a desired period of time, wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from group consisting of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml for desired period of time after injecting the composition comprising compound I, pharmaceutically acceptable salts or solvates thereof. In one embodiment, the intramuscular composition maintains blood plasma fulvestrant concentration selected from the group consisting of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml for a desired period of time, wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of at least 1 ng/ml for at least one week after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml, at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml, or at least 50 ng/ml for at least one week after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant at least 1 ng/ml for at least two weeks after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 2 weeks after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant at least 1 ng/ml for at least three weeks after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least three weeks after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant at least 1 ng/ml for at least four weeks after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least four weeks after injecting the composition comprising compound I.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant of at least 1 ng/ml for at least one month after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma concentration of fulvestrant selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least one month after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of at least 1 ng/ml for at least two months after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma Fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least two months after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of at least 1 ng/ml for at least three months after injecting the composition comprising compound I, pharmaceutically acceptable salts or solvates thereof. In one embodiment, the intramuscular composition comprising compound I, pharmaceutically acceptable salts or solvates thereof; and at least one pharmaceutically acceptable excipient, wherein the composition administered to a subject in need thereof maintains blood plasma Fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least three months after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 500 ng/ml for desired period of time after injecting the composition comprising compound I, pharmaceutically acceptable salts or solvates thereof. In one embodiment, the intramuscular composition maintains blood plasma fulvestrant concentration of about 1 ng/ml to about 500 ng/ml for a desired period of time, wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from group consisting of about 1 ng/ml to about 500 ng/ml, about 1 ng/ml to about 490 ng/ml, about 1 ng/ml to about 480 ng/ml, about 1 ng/ml to about 470 ng/ml, about 1 ng/ml to about 460 ng/ml, about 1 ng/ml to about 450 ng/ml, about 1 ng/ml to about 440 ng/ml, about 1 ng/ml to about 430 ng/ml, about 1 ng/ml to about 420 ng/ml, about 1 ng/ml to about 410 ng/ml, about 1 ng/ml to about 400 ng/ml, about 1 ng/ml to about 390 ng/ml, about 1 ng/ml to about 380 ng/ml, about 1 ng/ml to about 370 ng/ml, about 1 ng/ml to about 360 ng/ml, about 1 ng/ml to about 350 ng/ml, about 1 ng/ml to about 340 ng/ml, about 1 ng/ml to about 330 ng/ml, about 1 ng/ml to about 320 ng/ml, about 1 ng/ml to about 310 ng/ml, about 1 ng/ml to about 300 ng/ml, about 1 ng/ml to about 290 ng/ml, about 1 ng/ml to about 280 ng/ml, about 1 ng/ml to about 270 ng/ml, about 1 ng/ml to about 260 ng/ml, about 1 ng/ml to about 250 ng/ml, about 1 ng/ml to about 240 ng/ml, about 1 ng/ml to about 230 ng/ml, about 1 ng/ml to about 220 ng/ml, about 1 ng/ml to about 210 ng/ml, about 1 ng/ml to about 200 ng/ml, about 1 ng/ml to about 190 ng/ml, about 1 ng/ml to about 180 ng/ml, about 1 ng/ml to about 170 ng/ml, about 1 ng/ml to about 160 ng/ml, about 1 ng/ml to about 150 ng/ml, 1 ng/ml to about 140 ng/ml, about 1 ng/ml to about 130 ng/ml, about 1 ng/ml to about 120 ng/ml, about 1 ng/ml to about 110 ng/ml, about 1 ng/ml to about 100 ng/ml for desired period of time after injecting the composition comprising compound I, pharmaceutically acceptable salts or solvates thereof. In one embodiment, the intramuscular composition maintains blood plasma fulvestrant concentration selected from group consisting of about 1 ng/ml to about 500 ng/ml, about 1 ng/ml to about 490 ng/ml, about 1 ng/ml to about 480 ng/ml, about 1 ng/ml to about 470 ng/ml, about 1 ng/ml to about 460 ng/ml, about 1 ng/ml to about 450 ng/ml, about 1 ng/ml to about 440 ng/ml, about 1 ng/ml to about 430 ng/ml, about 1 ng/ml to about 420 ng/ml, about 1 ng/ml to about 410 ng/ml, about 1 ng/ml to about 400 ng/ml, about 1 ng/ml to about 390 ng/ml, about 1 ng/ml to about 380 ng/ml, about 1 ng/ml to about 370 ng/ml, about 1 ng/ml to about 360 ng/ml, about 1 ng/ml to about 350 ng/ml, about 1 ng/ml to about 340 ng/ml, about 1 ng/ml to about 330 ng/ml, about 1 ng/ml to about 320 ng/ml, about 1 ng/ml to about 310 ng/ml, about 1 ng/ml to about 300 ng/ml, about 1 ng/ml to about 290 ng/ml, about 1 ng/ml to about 280 ng/ml, about 1 ng/ml to about 270 ng/ml, about 1 ng/ml to about 260 ng/ml, about 1 ng/ml to about 250 ng/ml, about 1 ng/ml to about 240 ng/ml, about 1 ng/ml to about 230 ng/ml, about 1 ng/ml to about 220 ng/ml, about 1 ng/ml to about 210 ng/ml, about 1 ng/ml to about 200 ng/ml, about 1 ng/ml to about 190 ng/ml, about 1 ng/ml to about 180 ng/ml, about 1 ng/ml to about 170 ng/ml, about 1 ng/ml to about 160 ng/ml, about 1 ng/ml to about 150 ng/ml, about 1 ng/ml to about 140 ng/ml, about 1 ng/ml to about 130 ng/ml, about 1 ng/ml to about 120 ng/ml, about 1 ng/ml to about 110 ng/ml, or about 1 ng/ml to about 100 ng/ml for a desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for desired period of time after injecting the composition comprising compound I, pharmaceutically acceptable salts or solvates thereof. In one embodiment, the intramuscular composition maintains blood plasma fulvestrant concentration of about 1 ng/ml to about 100 ng/ml for a desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least one week after injecting the composition comprising compound I, pharmaceutically acceptable salts or solvates thereof. In one embodiment, the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least one week after injecting the composition comprising compound I.

In one embodiment, the present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least two weeks after injecting the composition comprising compound I, pharmaceutically acceptable salts or solvates thereof. In one embodiment, the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration is selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least two weeks after injecting the composition comprising compound I, pharmaceutically acceptable salts or solvates thereof.

In one the present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least three weeks after injecting the composition comprising compound I, pharmaceutically acceptable salts or solvates thereof. In one embodiment, the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least three weeks after injecting the composition comprising compound I, pharmaceutically acceptable salts or solvates thereof.

The present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least four weeks after injecting the composition comprising compound I, pharmaceutically acceptable salts or solvates thereof. In one embodiment, the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration s selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least four weeks after injecting the composition comprising compound I, pharmaceutically acceptable salts or solvates thereof.

The present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least one month after injecting the composition comprising compound I, pharmaceutically acceptable salts or solvates thereof. In one embodiment, the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least one month after injecting the composition comprising compound I, pharmaceutically acceptable salts or solvates thereof.

The present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least two months after injecting the composition comprising compound I, pharmaceutically acceptable salts or solvates thereof. In one embodiment, the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least for two months after injecting the composition comprising compound I, pharmaceutically acceptable salts or solvates thereof.

In one embodiment, the present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for at least three months after injecting the composition comprising compound I, pharmaceutically acceptable salts or solvates thereof. In one embodiment, the present invention provides the intramuscular injectable composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least three months after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration selected from the group consisting of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml for desired period of time after injecting the composition comprising compound I, pharmaceutically acceptable salts or solvates thereof. In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration selected from the group consisting of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for desired period of time after injecting the composition comprising compound I, pharmaceutically acceptable salts or solvates thereof. In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least one week after injecting the composition comprising compound I, pharmaceutically acceptable salts or solvates thereof.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least two weeks after injecting the composition comprising compound I, pharmaceutically acceptable salts or solvates thereof.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least three weeks after injecting the composition comprising compound I, pharmaceutically acceptable salts or solvates thereof.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least four weeks after injecting the composition comprising compound I, pharmaceutically acceptable salts or solvates thereof.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least one month after injecting the composition comprising compound I, pharmaceutically acceptable salts or solvates thereof.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least two months after injecting the composition comprising compound I, pharmaceutically acceptable salts or solvates thereof.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least three months after injecting the composition comprising compound I, pharmaceutically acceptable salts or solvates thereof.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 500 ng/ml for desired period of time after injecting the composition comprising compound I, pharmaceutically acceptable salts or solvates thereof. In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 500 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 100 ng/ml for desired period of time after injecting the composition comprising compound I, pharmaceutically acceptable salts or solvates thereof. In one embodiment, the present invention provides the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 100 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the intramuscular composition administered to a subject in need thereof has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least one week after injecting the composition comprising compound I, pharmaceutically acceptable salts or solvates thereof.

In one embodiment, the intramuscular composition has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; about 10 mg/ml to about 300 mg/ml maintains blood plasma concentration of fulvestrant of about 1 ng/ml to about 100 ng/ml at least two weeks after injecting the composition comprising compound I, pharmaceutically acceptable salts or solvates thereof.

In one embodiment, the intramuscular composition has concentration of compound I, pharmaceutically acceptable salts or solvates thereof; and at least one pharmaceutically acceptable excipient about, wherein the composition administered to a subject in need thereof has concentration of compound I, pharmaceutically acceptable salts or solvates thereof; about 10 mg/ml to about 300 mg/ml maintains blood plasma concentration of fulvestrant of about 1 ng/ml to about 100 ng/ml for at least three weeks after injecting the composition comprising compound I, pharmaceutically acceptable salts or solvates thereof.

In one embodiment, the intramuscular composition has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; about 10 mg/ml to about 300 mg/ml maintains blood plasma concentration of fulvestrant of about 1 ng/ml to about 100 ng/ml for at least four weeks after injecting the composition comprising compound I, pharmaceutically acceptable salts or solvates thereof.

In one embodiment, the intramuscular composition has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; about 10 mg/ml to about 300 mg/ml maintains blood plasma concentration of fulvestrant of about 1 ng/ml to about 100 ng/ml for at least one month after injecting the composition comprising compound I, pharmaceutically acceptable salts or solvates thereof.

In one embodiment, the intramuscular composition has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; about 10 mg/ml to about 300 mg/ml maintains blood plasma concentration of fulvestrant of about 1 ng/ml to about 100 ng/ml for at least two months after injecting the composition comprising compound I, pharmaceutically acceptable salts or solvates thereof.

In one embodiment, the intramuscular composition has concentration of compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; about 10 mg/ml to about 300 mg/ml maintains blood plasma concentration of fulvestrant of about 1 ng/ml to about 100 ng/ml for at least three months after injecting the composition comprising compound I, pharmaceutically acceptable salts or solvates thereof.

In one embodiment, the composition comprises at least one excipient, where in the excipient is selected from oil, solvent, surfactant, antioxidant, polymer or mixture thereof.

In one embodiment, an oil or vehicle is selected from the group consisting of structurally modified or hydrolysed coconut oil, castor oil, olive oil, soybean oil, safflower oil, triglycerides, octyl and decyl glycerate, ethyl oleate, glyceryl linoleate, ethyl linoleate, glyceryl oleate, medium chain glyceride (MCT), cholesteryl oleate/linoleate or a mixture thereof. In one embodiment, the oil or vehicle has as a proportion selected from group consisting of at least about 10%, at least about 20%, at least about 30%, at least about 40%, at least about 50%, at least about 60%, at least about 70%, at least about 80%, at least about 90% or at least about 95% w/v of its composition. In one embodiment, the oil or vehicle is present in an amount selected form about 1% to about 95% w/v of its composition. In one embodiment, the oil or vehicle is present in an amount selected form group consisting of at least 3% w/v, at least 5% w/v, at least 10% w/v, at least 15% w/v, at least 20% w/v, at least 25% w/v, at least 30% w/v, at least 35% w/v, at least 40% w/v, at least 45% w/v, at least 50% w/v, at least 55% w/v, at least 60% w/v, at least 65% w/v, at least 70% w/v, at least 75% w/v, at least 80% w/v, at least 85% w/v, at least 90% w/v or at least 95% w/v. In one embodiment concentrations of oil or vehicle present in the composition is selected from about 95% w/v or less, 90% w/v or less, about 85% w/v or less, 80% w/v or less, about 75% w/v or less, about 70% w/v or less, about 65% w/v or less, about 60% w/v or less, about 55% w/v or less, about 50% w/v or less, about 45% w/v or less, about 40% w/v or less, about 35% w/v or less, about 30% w/v or less, about 25% w/v or less, about 20% w/v or less, about 15% w/v or less; or about 10% w/v or less.

In one embodiment, the solvents can be selected from pharmaceutically acceptable polar protic and aprotic solvents or mixture thereof. The polar protic solvents include alkyl alcohols, ethanol, tert-butanol, benzyl alcohol, cetyl alcohol, ethylene glycol, propylene glycol, butylene glycol, glycerin, glycerol, polysorbates, for example polysorbate 20, polysorbate 40, and polysorbate 80, cyclodextrins (such as hydroxypropyl-β-cyclodextrin), polyalkylene glycols, such as polyethylene glycol (PEG), polyethylene glycol 200 (PEG 200), polyethylene glycol 300 (PEG 300), polyethylene glycol 400 (PEG 400), polyethylene glycol 600 (PEG 600), polypropylene glycol, and polybutylene glycol, and primary amides such as niacinamide. The pharmaceutically acceptable polar aprotic solvents include N-methylpyrrolidone, ethyl acetate, dimethyl sulfoxide (DMSO), secondary and tertiary amides, wherein secondary amides are selected from N-ethylacetamide, N-ethylformamide, and tertiary amides are selected from dimethylacetamide (DMA), N-methyl-N-vinylacetamide, N,N-dimethylpropionamide, N,N-diethylacetamide (DEA), N,N-diisopropylformamide, N-methylpyrrolidine and N,N-dimethyl formamide. In one embodiment, solvents are also used as solubilizer.

In one embodiment, the solvent is present in an amount selected form about 1% to about 70% w/v of its composition. In one embodiment, the solvent is present in an amount selected form group consisting of at least 3% w/v, at least 5% w/v, at least 10% w/v, at least 15% w/v, at least 20% w/v, at least 25% w/v, at least 30% w/v, at least 35% w/v, at least 40% w/v, at least 45% w/v, at least 50% w/v, at least 55% w/v, at least 60% w/v, at least 65% w/v, or at least 70% w/v.

In one embodiment, the concentrations of a pharmaceutically acceptable solvent present in the composition are selected from at least 3% w/v, at least 5% w/v, at least 7% w/v, at least 10% w/v, at least 11% w/v, at least 12% w/v, at least 13% w/v, at least 14% w/v, at least 15% w/v or at least 16% w/v.

In one embodiment concentrations of pharmaceutically-acceptable solvent present in the composition is selected from about 38% w/v or less, about 36% w/v or less, about 34% w/v or less, about 32% w/v or less, about 30% w/v or less, about 28% w/v or less, about 26% w/v or less, about 24% w/v or less, about 22% w/v or less, about 20% w/v or less, about 18% w/v or less, about 16% w/v or less, about 14% w/v or less, about 12% w/v or less, about 10% w/v or less. In one embodiment, pharmaceutically-acceptable solvent present in any of the above composition is selected from the range of any minimum or maximum value described herein as 3-35% w/v, 4-35% w/v, 5-35% w/v, 5-32% w/v, 7-32% w/v, 10-30% w/v, 12-28% w/v, 15-25% w/v, 17-23% w/v, 18-22% w/v or 19-21% w/v.

In one embodiment concentrations of pharmaceutically-acceptable alcohol present in the composition is selected from about 38% w/v or less, about 36% w/v or less, about 34% w/v or less, about 32% w/v or less, about 30% w/v or less, about 28% w/v or less, about 26% w/v or less, about 24% w/v or less, about 22% w/v or less, about 20% w/v or less, about 18% w/v or less, about 16% w/v or less, about 14% w/v or less, about 12% w/v or less, about 10% w/v or less. In one embodiment, pharmaceutically-acceptable alcohol present in any of the above composition is selected from the range of any minimum or maximum value selected from group consisting of 3-35% w/v, 4-35% w/v, 5-35% w/v, 5-32% w/v, 7-32% w/v, 10-30% w/v, 12-28% w/v, 15-25% w/v, 17-23% w/v, 18-22% w/v or 19-21% w/v.

The pharmaceutically-acceptable non-aqueous ester solvent may consist of one or a mixture of two or more pharmaceutically-acceptable non-aqueous ester solvents. In one embodiment pharmaceutically-acceptable non-aqueous ester solvent is selected from benzyl benzoate, ethyl oleate, ethyl acetate isopropyl myristate, isopropyl palmitate or a mixture thereof.

In one embodiment, concentrations of the pharmaceutically acceptable non-aqueous ester solvent present in any of the composition of the present invention is selected from at least about 5% w/v, at least about 8% w/v, at least about 10% w/v, at least about 11% w/v, at least about 12% w/v, at least about 13% w/v, at least about 15% w/v, at least about 16% w/v, at least about 17% w/v, at least about 18% w/v, at least about 19% w/v or at least about 20% w/v. In one embodiment, the maximal concentrations of the pharmaceutically acceptable non-aqueous ester solvent are selected from about 60% w/v or less, about 50% w/v or less, about 45% w/v or less, about 40% w/v or less, about 35% w/v or less, about 30% w/v or less and about 25% w/v or less. In one embodiment, the ranges of pharmaceutically-acceptable non-aqueous ester solvent present in any of the composition of the present invention is selected from 1-60% w/v, 5-60% w/v, 7-55% w/v, 8-50% w/v, 10-50% w/v, 10-45% w/v, 10-40% w/v, 10-35% w/v, 10-30% w/v, 10-25% w/v, 12-25% w/v, 12-22% w/v, 12-20% w/v, 12-18% w/v, 13-17% w/v or 14-16% w/v.

In one embodiment, the surfactants is selected form non-ionic, ionic surfactants such as ethylene, propylene oxide, sorbitan esters (e.g., sorbitan monolaurate), ethoxylates, and copolymers.

Examples of commercially available ionic surfactants are sulphates (anionic) or ester sulphonates, quaternary ammonium salts (cationic), Poloxamer 188, and fatty acids. In one embodiment, the surfactant is present in an amount selected form about 0.01% to about 50% w/v of its composition.

In one embodiment, the surfactant has as a proportion selected from group of about 0.01% to about 1% w/v, about 0.01% to about 2% w/v, about 0.01% to about 3% w/v, about 0.01% to about 4% w/v, about 0.01% to about 5% w/v, about 0.01% to about 6% w/v, about 0.01% to about 7% w/v, about 0.01% to about 8% w/v, about 0.01% to about 9% w/v, about 0.01% to about 10% w/v, about 0.01% to about 20% w/v, about 0.01% to about 30% w/v, about 0.01% to about 40% w/v or about 0.01% to about 50% w/v of its composition.

In one embodiment, the antioxidant is selected from antioxidants that can be employed are Alpha-Tocopherol, Ascorbic acid, Ascorbyl palmitate, Thioglycerol and its derivatives, Sodium bisulphate, Sodium metabisulphite, Sodium formaldehyde sulphoxylate, Thiourea, Ascorbic acid ester, BHT (Butylated hydroxyl toluene), Tocopherols, Lipoic acid, Alpha-lipoic acid, Dihydro lipoic acid. In one embodiment, the antioxidant is present in an amount selected form about 0.01% to about 50% w/v of its composition. In one embodiment, the surfactant has as a proportion selected from group of about 0.01% to about 1% w/v, about 0.01% to about 2% w/v, about 0.01% to about 3% w/v, about 0.01% to about 4% w/v, about 0.01% to about 5% w/v, about 0.01% to about 6% w/v, about 0.01% to about 7% w/v, about 0.01% to about 8% w/v, about 0.01% to about 9% w/v, about 0.01% to about 10% w/v, about 0.01% to about 12% w/v, about 0.01% to about 14% w/v, about 0.01% to about 16% w/v, about 0.01% to about 18% w/v, about 0.01% to about 20% w/v of its composition, about 0.01% to about 22% w/v, about 0.01% to about 24% w/v, about 0.01% to about 26% w/v, about 0.01% to about 28% w/v, about 0.01% to about 30% w/v, about 0.01% to about 32% w/v, about 0.01% to about 34% w/v, about 0.01% to about 36% w/v, about 0.01% to about 38% w/v, about 0.01% to about 40% w/v, about 0.01% to about 42% w/v, about 0.01% to about 44% w/v, about 0.01% to about 46% w/v, about 0.01% to about 48% w/v or about 0.01% to about 50% w/v of its composition.

In one embodiment, the polymer is selected from Poly(D,L-lactide) (DL-PLA), Poly (D,L-lactide-co-glycolide) (PLGA) in different proportion like 50:50, 65:35, 75:25, 85:15 and with different end group like ester (uncapped) or carboxylic acid terminated Poly(D,L glycolide) DL-PLG with different proportion like 50:50, 65:35, 75:25, 85:15, PDLG (50:50) and PDLG (75:25), DL-PLA, Poly(L-lactide) (L-PLA), Poly (D,L-lactide-co-glycolide)-polyethylene glycol (PLGA-PEG) conjugated, Poly (D,L-lactide-co-glycolide)-polycaprolactone (PLGA-PCL) conjugated, Poly (D,L-lactide-co-glycolide)-Poly(L-lactide) (PLGA-PLL) conjugated, Carboxy methyl cellulose, sucrose acetate isobutyrate, PLGA-PEG-PLGA Poly (D,L-lactide-co-glycolide)-polyethylene glycol-poly (D,L-lactide-co-glycolide). In one embodiment, the polymer is selected from group consisting of at least about 1%, at least about 5, % at least about 10%, at least about 20%, at least about 30%, at least about 40%, at least about 50%, at least about 60%, at least about 70%, at least about 80%, at least about 90% or at least about 95% w/v of its composition. In one embodiment, the polymer is selected from about 1% to about 80% w/v of its composition. In one embodiment, the concentrations of the polymer present in the composition is selected from at least 3% w/v, at least 5% w/v, at least 10% w/v, at least 15% w/v, at least 20% w/v, at least 25% w/v, at least 30% w/v, at least 35% w/v, at least 40% w/v, at least 45% w/v, at least 50% w/v, at least 55% w/v, at least 60% w/v, at least 65% w/v, at least 70% w/v, at least 75% w/v or at least 80% w/v. In one embodiment concentrations of the polymer present in the composition is selected from about 80% w/v or less, about 75% w/v or less, about 70% w/v or less, about 65% w/v or less, about 60% w/v or less, about 55% w/v or less, about 50% w/v or less, about 45% w/v or less, about 40% w/v or less, about 35% w/v or less, about 30% w/v or less, about 25% w/v or less, about 20% w/v or less, about 15% w/v or less; or about 10% w/v or less.

In one embodiment, the present invention provides low volume injection comprising composition having compound I, pharmaceutically acceptable salts or solvates thereof; and at least one pharmaceutically acceptable excipient. In one embodiment, the present invention provides the intramuscular injectable composition comprising the compound I, pharmaceutically acceptable salts or solvates thereof; and at least one pharmaceutically acceptable excipient, wherein the injectable volume administered through intramuscular route is about 1 ml to about 10 ml. In one embodiment, the present invention provides low volume injection comprising composition having compound I, pharmaceutically acceptable salts or solvates thereof; and at least one pharmaceutically acceptable excipient. In one embodiment, the present invention provides the intramuscular injectable composition comprising the compound I, pharmaceutically acceptable salts or solvates thereof; and at least one pharmaceutically acceptable excipient, wherein the injectable volume administered through intramuscular route is about 0.1 ml to about 10 ml. In one embodiment, the injectable volume administered through intramuscular route is selected from about 1 ml to about 9 ml, about 1 ml to about 8 ml, about 1 ml to about 7 ml, about 1 ml to about 6 ml, about 1 ml to about 5 ml, about 1 ml to about 4 ml, about 1 ml to about 3 ml or about 1 ml to about 2 ml. In one embodiment, the injectable volume administered through intramuscular route of composition is selected from about 0.1 ml to about 9 ml, about 0.1 ml to about 8 ml, about 0.1 ml to about 7 ml, about 0.1 ml to about 6 ml, about 0.1 ml to about 5 ml, about 0.1 ml to about 4 ml, about 0.1 ml to about 3 ml or about 0.1 ml to about 2 ml. In one embodiment, the injectable volume administered through intramuscular route of composition is selected from about 1 ml, about 2 ml, about 3 ml, about 4 ml, about 5 ml, about 6 ml, about 7 ml, about 8, ml, about 9 ml or about 10 ml. In one embodiment, the injectable volume administered through intramuscular route of composition is selected from about 0.1 ml, about 0.2 ml, about 0.3 ml, about 0.4 ml, about 0.5 ml, about 1.5 ml, about 2.5, about 3.3 ml, about 3.5 ml, about 4.5 ml, about 5.5 ml, about 6.5 ml, about 7.5 ml, about 8.5, ml, or about 9.5 ml.

In one embodiment, the invention provides pharmaceutical composition intended for intramuscular injection in a divided dose of compound I, pharmaceutically acceptable salts or solvates thereof; and at least one pharmaceutically acceptable excipient. In one embodiment, the divided dose of composition comprising compound I, pharmaceutically acceptable salts or solvates thereof; and at least one pharmaceutically acceptable excipient is equally divided dose and given at two different sites of injection. In one embodiment, the present invention includes composition comprising compound I, pharmaceutically acceptable salts or solvates thereof; and at least one pharmaceutically acceptable excipient which achieves the blood plasma fulvestrant concentration with single or equally divided dose of composition.

In one embodiment, the invention provides the method for the treatment of a benign or malignant disease of the breast or reproductive comprising use of compound I, pharmaceutically acceptable salts or solvates thereof; and at least one pharmaceutically acceptable excipient and optionally in combination with therapeutic agent selected from aromatase inhibitor, CDK-4/6 inhibitor, PI3K (Phosphatidylinositol-3-kinase) inhibitor or mammalian target of rapamycin (mTOR) inhibitor.

In one embodiment, the aromatase inhibitor is selected from aminoglutethimide, testolactone, anastrozole, letrozole, exemestane, vorozole, formestane, fadrozole, 1,4,6-Androstatrien-3,17-dione, femara, and 4-Androstene-3,6,17-trione.

In one embodiment, the CDK-4 inhibitor is selected from palbociclib, ribociclib, and abemaciclib.

In one embodiment, the PI3K (Phosphatidylinositol-3-kinase) inhibitor is selected from alpelisib.

In one embodiment, the Mammalian target of rapamycin (mTOR) inhibitor is selected from Everolimus.

A further feature of the invention is use of compound I, pharmaceutically acceptable salts or solvates thereof; and at least one pharmaceutically acceptable excipient in the preparation of a pharmaceutical composition as describe herein above, for the treatment of a benign or malignant disease of the breast or reproductive tract, preferably treating breast cancer.

In one embodiment, the composition of the present invention is used for the hormonal dependent benign or malignant disease of the breast or reproductive tract.

In one embodiment, the hormonal dependent benign or malignant disease of the breast is breast cancer.

In one embodiment, the present invention provides a method for treating a hormonal dependent benign or malignant disease of the breast or reproductive tract comprising administering intramuscularly to a subject in need of such treatment the compound I

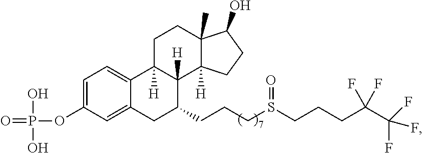

pharmaceutically acceptable salts or solvates thereof; and at least one pharmaceutically acceptable excipient.

In one embodiment, the present invention provides a method for treating a hormonal dependent benign or malignant disease of the breast or reproductive tract comprising administering intramuscularly to a subject in need of such treatment the compound I

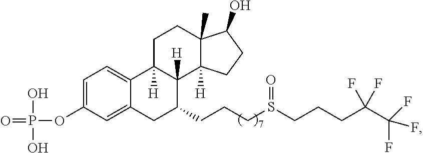

pharmaceutically acceptable salts or solvates thereof; and at least one pharmaceutically acceptable excipient, wherein at least one pharmaceutically acceptable excipient is selected from oil, surfactant, solvent, polymer or mixture thereof.

In one embodiment, the present invention provides a method for treating a hormonal dependent benign or malignant disease of the breast or reproductive tract comprising administering intramuscularly to a subject in need of such treatment the compound I

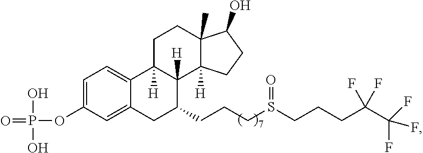

pharmaceutically acceptable salts or solvates thereof; and at least one pharmaceutically acceptable excipient; wherein the composition administered to a subject in need thereof comprises about 10 mg to about 1000 mg of compound I, pharmaceutically acceptable salts or solvates thereof. In one embodiment, the present invention provides a method for treating a hormonal dependent benign or malignant disease of the breast or reproductive tract comprising administering intramuscularly to a subject in need of such treatment the compound I, pharmaceutically acceptable salts or solvates thereof; and at least one pharmaceutically acceptable excipient; wherein the composition administered to a subject in need thereof comprises compound I, pharmaceutically acceptable salts or solvates thereof selected from group consisting of 50 mg to about 1000 mg, 100 mg to about 1000 mg, 150 mg to about 1000 mg, 50 mg to about 1000 mg, 100 mg to about 800 mg, 150 mg to about 800 mg, 50 mg to about 600 mg, 100 mg to about 600 mg, 150 mg to about 600 mg, 50 mg to about 500 mg, 100 mg to about 500 mg, 150 mg to about 500 mg.

In one embodiment, the present invention provides a method for treating a hormonal dependent benign or malignant disease of the breast or reproductive tract comprising administering intramuscularly to a subject in need of such treatment the compound I, pharmaceutically acceptable salts or solvates thereof; and at least one pharmaceutically acceptable excipient; wherein the composition administered to a subject in need thereof comprises compound I, pharmaceutically acceptable salts or solvates thereof selected from group consisting of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg.

In one embodiment, the present invention provides a method for treating a hormonal dependent benign or malignant disease of the breast or reproductive tract comprising administering intramuscularly to a subject in need of such treatment the composition having compound I

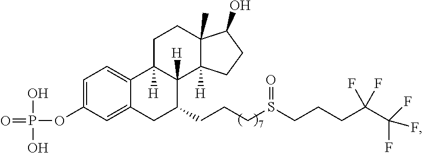

pharmaceutically acceptable salts or solvates thereof; and at least one pharmaceutically acceptable excipient, wherein the composition administered to a subject in need thereof has concentration of compound I, pharmaceutically acceptable salts or solvates thereof from about 10 mg/ml to about 300 mg/ml.

In one embodiment, the present invention provides a method for treating a hormonal dependent benign or malignant disease of the breast or reproductive tract comprising administering intramuscularly to a subject in need of such treatment the composition having compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof selected from about 20 mg/ml to about 300 mg/ml, about 30 mg/ml to about 300 mg/ml, about 40 mg/ml to about 300 mg/ml, about 50 mg/ml to about 300 mg/ml, about 60 mg/ml to about 300 mg/ml, about 70 mg/ml to about 300 mg/ml, about 80 mg/ml to about 300 mg/ml, about 90 mg/ml to about 300 mg/ml, about 100 mg/ml to about 300 mg/ml, about 110 mg/ml to about 300 mg/ml, about 120 mg/ml to about 300 mg/ml, about 130 mg/ml to about 300 mg/ml, about 140 mg/ml to about 300 mg/ml, about 150 mg/ml to about 300 mg/ml, about 160 mg/ml to about 300 mg/ml, about 170 mg/ml to about 300 mg/ml, about 180 mg/ml to about 300 mg/ml, about 190 mg/ml to about 300 mg/ml, about 200 mg/ml to about 300 mg/ml, about 210 mg/ml to about 300 mg/ml, about 220 mg/ml to about 300 mg/ml, about 230 mg/ml to about 300 mg/ml, about 240 mg/ml to about 300 mg/ml, about 250 mg/ml to about 300 mg/ml, about 260 mg/ml to about 300 mg/ml, about 270 mg/ml to about 300 mg/ml, about 280 mg/ml to about 300 mg/ml, or about 290 mg/ml to about 300 mg/ml.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient wherein the composition administered to a subject in need thereof has concentration of compound I, pharmaceutically acceptable salts or solvates thereof, about 10 mg/ml.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient wherein the composition administered to a subject in need thereof has concentration of compound I, pharmaceutically acceptable salts or solvates thereof selected from about 20 mg/ml, about 30 mg/ml, about 40 mg/ml, about 50 mg/ml, about 60 mg/ml, about 70, mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml, about 260 mg/ml, about 270 mg/ml, about 280 mg/ml, about 290 mg/ml or about 300 mg/ml.

In one embodiment the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration for a desired period of time after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from the group consisting of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from the group consisting of at least 1 ng/ml, at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml for desired period of time after injecting the composition comprising compound I, pharmaceutically acceptable salts or solvates thereof, wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of at least 1 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of at least 1 ng/ml for desired period of time after injecting the composition comprising compound I, pharmaceutically acceptable salts or solvates thereof, wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I

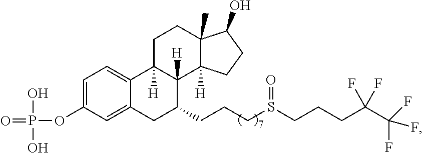

pharmaceutically acceptable salts or solvates thereof; and at least one pharmaceutically acceptable excipient, wherein the composition administered to a subject in need thereof maintains blood plasma fulvestrant concentration of at least 1 ng/ml for at least one week after injecting the composition comprising compound I, pharmaceutically acceptable salts or solvates thereof; In one embodiment, the composition comprising compound I, pharmaceutically acceptable salts or solvates thereof when administered to a subject in need thereof maintains blood plasma fulvestrant concentration of at least 1 ng/ml selected from the time period of least two weeks, at least three weeks, at least four weeks, at least one month, at least two months or at least three months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient wherein the composition administered to a subject in need thereof maintains blood plasma concentration of fulvestrant of at least 1 ng/ml for at least one week after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof and at least one pharmaceutically acceptable excipient wherein the composition administered to a subject in need thereof maintains a blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml, at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least one week.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of at least 1 ng/ml for at least two weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition maintains blood plasma fulvestrant concentration of selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 2 weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I maintains blood plasma fulvestrant concentration of at least 1 ng/ml for at least three weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least three weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I maintains blood plasma fulvestrant concentration of at least 1 ng/ml for at least four weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least four weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of at least 1 ng/ml for at least one month after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least one month after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of at least 1 ng/ml for at least two months after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least two months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of at least 1 ng/ml for at least three months after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least three months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 500 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 500 ng/ml for desired period of time after injecting the composition comprising compound I, pharmaceutically acceptable salts or solvates thereof, wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from group consisting of about 1 ng/ml to about 500 ng/ml, about 1 ng/ml to about 490 ng/ml, about 1 ng/ml to about 480 ng/ml, 1 ng/ml to about 470 ng/ml, about 1 ng/ml to about 460 ng/ml, about 1 ng/ml to about 450 ng/ml, 1 ng/ml to about 440 ng/ml, about 1 ng/ml to about 430 ng/ml, about 1 ng/ml to about 420 ng/ml, 1 ng/ml to about 410 ng/ml, about 1 ng/ml to about 400 ng/ml, about 1 ng/ml to about 390 ng/ml, about 1 ng/ml to about 380 ng/ml, 1 ng/ml to about 370 ng/ml, about 1 ng/ml to about 360 ng/ml, about 1 ng/ml to about 350 ng/ml, 1 ng/ml to about 340 ng/ml, about 1 ng/ml to about 330 ng/ml, about 1 ng/ml to about 320 ng/ml, 1 ng/ml to about 310 ng/ml, about 1 ng/ml to about 300 ng/ml, about 1 ng/ml to about 290 ng/ml, about 1 ng/ml to about 280 ng/ml, 1 ng/ml to about 270 ng/ml, about 1 ng/ml to about 260 ng/ml, about 1 ng/ml to about 250 ng/ml, 1 ng/ml to about 240 ng/ml, about 1 ng/ml to about 230 ng/ml, about 1 ng/ml to about 220 ng/ml, 1 ng/ml to about 210 ng/ml, about 1 ng/ml to about 200 ng/ml, about 1 ng/ml to about 190 ng/ml, about 1 ng/ml to about 180 ng/ml, 1 ng/ml to about 170 ng/ml, about 1 ng/ml to about 160 ng/ml, about 1 ng/ml to about 150 ng/ml, 1 ng/ml to about 140 ng/ml, about 1 ng/ml to about 130 ng/ml, about 1 ng/ml to about 120 ng/ml, 1 ng/ml to about 110 ng/ml, 1 ng/ml to about 100 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from group consisting of about 1 ng/ml to about 500 ng/ml, about 1 ng/ml to about 490 ng/ml, about 1 ng/ml to about 480 ng/ml, 1 ng/ml to about 470 ng/ml, about 1 ng/ml to about 460 ng/ml, about 1 ng/ml to about 450 ng/ml, 1 ng/ml to about 440 ng/ml, about 1 ng/ml to about 430 ng/ml, about 1 ng/ml to about 420 ng/ml, 1 ng/ml to about 410 ng/ml, about 1 ng/ml to about 400 ng/ml, about 1 ng/ml to about 390 ng/ml, about 1 ng/ml to about 380 ng/ml, 1 ng/ml to about 370 ng/ml, about 1 ng/ml to about 360 ng/ml, about 1 ng/ml to about 350 ng/ml, 1 ng/ml to about 340 ng/ml, about 1 ng/ml to about 330 ng/ml, about 1 ng/ml to about 320 ng/ml, 1 ng/ml to about 310 ng/ml, about 1 ng/ml to about 300 ng/ml, about 1 ng/ml to about 290 ng/ml, about 1 ng/ml to about 280 ng/ml, 1 ng/ml to about 270 ng/ml, about 1 ng/ml to about 260 ng/ml, about 1 ng/ml to about 250 ng/ml, 1 ng/ml to about 240 ng/ml, about 1 ng/ml to about 230 ng/ml, about 1 ng/ml to about 220 ng/ml, 1 ng/ml to about 210 ng/ml, about 1 ng/ml to about 200 ng/ml, about 1 ng/ml to about 190 ng/ml, about 1 ng/ml to about 180 ng/ml, 1 ng/ml to about 170 ng/ml, about 1 ng/ml to about 160 ng/ml, about 1 ng/ml to about 150 ng/ml, 1 ng/ml to about 140 ng/ml, about 1 ng/ml to about 130 ng/ml, about 1 ng/ml to about 120 ng/ml, 1 ng/ml to about 110 ng/ml, 1 ng/ml to about 100 ng/ml for desired period of time after injecting the composition comprising compound I, pharmaceutically acceptable salts or solvates thereof, wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of about 1 ng/ml to about 100 ng/ml for desired period of time after injecting the composition comprising compound I, pharmaceutically acceptable salts or solvates thereof, wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I

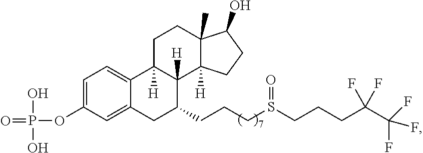

5, pharmaceutically acceptable salts or solvates thereof; and at least one pharmaceutically acceptable excipient; wherein the composition administered to a subject in need thereof maintains blood plasma fulvestrant concentration of about 1 ng/ml to about 100 ng/ml for at least one week after injecting the composition comprising compound I, pharmaceutically acceptable salts or solvates thereof. In one embodiment, the composition comprising compound I, pharmaceutically acceptable salts or solvates thereof; when administered to a subject in need thereof maintains blood plasma fulvestrant concentration of about 1 ng/ml to about 100 ng/ml selected from the time of least two weeks, at least three weeks, at least four weeks, at least one month, at least two months or at least three months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein, wherein the composition administered to a subject in need thereof maintains blood plasma fulvestrant concentration of about 1 ng/ml to about 100 ng/ml for at least one week after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration of fulvestrant selected from about 2 ng/ml to about 100 ng/ml, 4 ng/ml to about 100 ng/ml, 6 ng/ml to about 100 ng/ml, 8 ng/ml to about 100 ng/ml, 10 ng/ml to about 100 ng/ml, 12 ng/ml to about 100 ng/ml, 14 ng/ml to about 100 ng/ml, 16 ng/ml to about 100 ng/ml, 18 ng/ml to about 100 ng/ml, 20 ng/ml to about 100 ng/ml, 22 ng/ml to about 100 ng/ml, 24 ng/ml to about 100 ng/ml, 26 ng/ml to about 100 ng/ml, 28 ng/ml to about 100 ng/ml or about 30 ng/ml to about 100 ng/ml for at least one week after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma concentration of fulvestrant of about 1 ng/ml to about 100 ng/ml for at least two weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein, wherein the composition administered to a subject in need thereof maintains blood plasma fulvestrant concentration selected from about 2 ng/ml to about 100 ng/ml, 4 ng/ml to about 100 ng/ml, 6 ng/ml to about 100 ng/ml, 8 ng/ml to about 100 ng/ml, 10 ng/ml to about 100 ng/ml, 12 ng/ml to about 100 ng/ml, 14 ng/ml to about 100 ng/ml, 16 ng/ml to about 100 ng/ml, 18 ng/ml to about 100 ng/ml, 20 ng/ml to about 100 ng/ml, 22 ng/ml to about 100 ng/ml, 24 ng/ml to about 100 ng/ml, 26 ng/ml to about 100 ng/ml, 28 ng/ml to about 100 ng/ml or about 30 ng/ml to about 100 ng/ml for at least two weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of about 1 ng/ml to about 100 ng/ml for at least three weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least three weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of about 1 ng/ml to about 100 ng/ml for at least four weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least four weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof maintains blood plasma fulvestrant concentration of about 1 ng/ml to about 100 ng/ml for at least one month after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least one month after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof maintains blood plasma fulvestrant concentration of about 1 ng/ml to about 100 ng/ml for at least two months after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least for two months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition comprising compound I, pharmaceutically acceptable salts or solvates thereof; maintains blood plasma fulvestrant concentration of at least about 1 ng/ml to 100 ng/ml for at least three months after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof maintains blood plasma fulvestrant concentration selected from about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml for at least three months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I

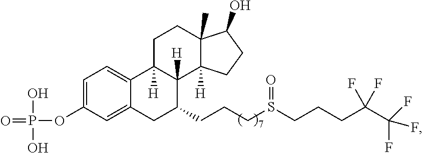

pharmaceutically acceptable salts or solvates thereof; and at least one pharmaceutically acceptable excipient; wherein the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least one week after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof; from 10 mg/ml to 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 1 week after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I from about 10 mg/ml to about 300 mg/ml and attains a maintains blood plasma fulvestrant concentration of at least 1 ng/ml for at least two weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof; from about 10 mg/ml to about 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 2 weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I from about 10 mg/ml to about 300 mg/ml and maintains blood plasma fulvestrant concentration of at least 1 ng/ml for at least three weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof from about 10 mg/ml to about 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 3 weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof from about 10 mg/ml to about 300 mg/ml and maintains blood plasma fulvestrant concentration of at least 1 ng/ml for at least four weeks after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof from about 10 mg/ml to about 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 4 weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for at least one month after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof; from about 10 mg/ml to about 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least one month after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof from about 10 mg/ml to about 300 mg/ml and maintains blood plasma fulvestrant concentration of at least 1 ng/ml for at least two months after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof; from about 10 mg/ml to about 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least two months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof from about 10 mg/ml to about 300 mg/ml and maintains blood plasma fulvestrant concentration of at least 1 ng/ml for at least three months after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient; wherein the composition administered to a subject in need thereof has concentration of compound I, pharmaceutically acceptable salts or solvates thereof; from about 10 mg/ml to about 300 mg/ml and maintains blood plasma fulvestrant concentration selected from at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml or at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48, at least 49 ng/ml or at least 50 ng/ml for at least 3 months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 500 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 500 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration selected from group consisting of about 1 ng/ml to about 500 ng/ml, about 1 ng/ml to about 490 ng/ml, about 1 ng/ml to about 480 ng/ml, about 1 ng/ml to about 470 ng/ml, about 1 ng/ml to about 460 ng/ml, about 1 ng/ml to about 450 ng/ml, about 1 ng/ml to about 440 ng/ml, about 1 ng/ml to about 430 ng/ml, about 1 ng/ml to about 420 ng/ml, about 1 ng/ml to about 410 ng/ml, about 1 ng/ml to about 400 ng/ml, about 1 ng/ml to about 390 ng/ml, about 1 ng/ml to about 380 ng/ml, about 1 ng/ml to about 370 ng/ml, about 1 ng/ml to about 360 ng/ml, about 1 ng/ml to about 350 ng/ml, about 1 ng/ml to about 340 ng/ml, about 1 ng/ml to about 330 ng/ml, about 1 ng/ml to about 320 ng/ml, about 1 ng/ml to about 310 ng/ml, about 1 ng/ml to about 300 ng/ml, about 1 ng/ml to about 290 ng/ml, about 1 ng/ml to about 280 ng/ml, about 1 ng/ml to about 270 ng/ml, about 1 ng/ml to about 260 ng/ml, about 1 ng/ml to about 250 ng/ml, about 1 ng/ml to about 240 ng/ml, about 1 ng/ml to about 230 ng/ml, about 1 ng/ml to about 220 ng/ml, about 1 ng/ml to about 210 ng/ml, about 1 ng/ml to about 200 ng/ml, about 1 ng/ml to about 190 ng/ml, about 1 ng/ml to about 180 ng/ml, about 1 ng/ml to about 170 ng/ml, about 1 ng/ml to about 160 ng/ml, about 1 ng/ml to about 150 ng/ml, about 1 ng/ml to about 140 ng/ml, about 1 ng/ml to about 130 ng/ml, about 1 ng/ml to about 120 ng/ml, about 1 ng/ml to about 110 ng/ml, about 1 ng/ml to about 100 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration selected from group consisting of about 1 ng/ml to about 500 ng/ml, about 1 ng/ml to about 490 ng/ml, about 1 ng/ml to about 480 ng/ml, about 1 ng/ml to about 470 ng/ml, about 1 ng/ml to about 460 ng/ml, about 1 ng/ml to about 450 ng/ml, about 1 ng/ml to about 440 ng/ml, about 1 ng/ml to about 430 ng/ml, about 1 ng/ml to about 420 ng/ml, about 1 ng/ml to about 410 ng/ml, about 1 ng/ml to about 400 ng/ml, about 1 ng/ml to about 390 ng/ml, about 1 ng/ml to about 380 ng/ml, 1 ng/ml to about 370 ng/ml, about 1 ng/ml to about 360 ng/ml, about 1 ng/ml to about 350 ng/ml, 1 ng/ml to about 340 ng/ml, about 1 ng/ml to about 330 ng/ml, about 1 ng/ml to about 320 ng/ml, 1 ng/ml to about 310 ng/ml, about 1 ng/ml to about 300 ng/ml, about 1 ng/ml to about 290 ng/ml, about 1 ng/ml to about 280 ng/ml, about 1 ng/ml to about 270 ng/ml, about 1 ng/ml to about 260 ng/ml, about 1 ng/ml to about 250 ng/ml, about 1 ng/ml to about 240 ng/ml, about 1 ng/ml to about 230 ng/ml, about 1 ng/ml to about 220 ng/ml, about 1 ng/ml to about 210 ng/ml, about 1 ng/ml to about 200 ng/ml, about 1 ng/ml to about 190 ng/ml, about 1 ng/ml to about 180 ng/ml, about 1 ng/ml to about 170 ng/ml, about 1 ng/ml to about 160 ng/ml, about 1 ng/ml to about 150 ng/ml, about 1 ng/ml to about 140 ng/ml, about 1 ng/ml to about 130 ng/ml, about 1 ng/ml to about 120 ng/ml, about 1 ng/ml to about 110 ng/ml, about 1 ng/ml to about 100 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 100 ng/ml for desired period of time after injecting the composition comprising compound I, pharmaceutically acceptable salts or solvates thereof. In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 100 ng/ml for desired period of time after injecting the composition comprising compound I, pharmaceutically acceptable salts or solvates thereof; wherein the desired period of time is selected from group consisting of at least one week, at least two weeks, at least three weeks, at least four weeks, at least one month, at least two months or at least three months.

In one embodiment, the present invention provides a method for treating a hormonal dependent benign or malignant disease of the breast or reproductive tract comprising administering intramuscularly to a subject in need the intramuscular composition comprising compound I

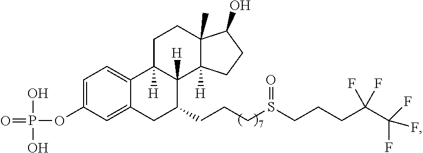

pharmaceutically acceptable salts or solvates thereof; and at least one pharmaceutically acceptable excipient; wherein the intramuscular composition administered to a subject in need thereof has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least one week after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition has concentration of compound I about 10 mg/ml to about 300 mg/ml and maintains blood plasma fulvestrant concentration of about 1 ng/ml to about 100 ng/ml at least two weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least three weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least four weeks after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least one month after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least two months after injecting the composition comprising compound I, pharmaceutically acceptable salts or solvates thereof.

In one embodiment, the present invention provides a method for treating a hormonal dependent benign or malignant disease of the breast or reproductive tract comprising administering intramuscularly to a subject in need the intramuscular composition comprising compound I, pharmaceutically acceptable salts or solvates thereof; and at least one pharmaceutically acceptable excipient, wherein the composition has concentration of compound I, pharmaceutically acceptable salts or solvates thereof; about 10 mg/ml to about 300 mg/ml and maintains blood plasma fulvestrant concentration of about 1 ng/ml to about 100 ng/ml for at least three months after injecting the composition comprising compound I, pharmaceutically acceptable salts or solvates thereof.

In one embodiment, the present invention provides the intramuscular composition of compound I, pharmaceutically acceptable salts or solvates thereof; and at least one pharmaceutically acceptable excipient, wherein the composition is able to deliver Cmax of the fulvestrant as compared to standard fulvestrant injectable preparation in the ratio of 10:1 after injecting the composition comprising compound I, pharmaceutically acceptable salts or solvates thereof. In one embodiment, the present invention provides the intramuscular composition of compound I, pharmaceutically acceptable salts or solvates thereof; and at least one pharmaceutically acceptable excipient, wherein the composition is able to deliver Cmax of the fulvestrant as compared to standard fulvestrant injectable preparation in the ratio of 40:1 after injecting the composition comprising compound I, pharmaceutically acceptable salts or solvates thereof. In one embodiment the ratio of Cmax obtained from the composition comprising compound I, pharmaceutically acceptable salts or solvates thereof; in comparison to standard Fulvestrant injectable preparation can be selected from 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1 after injecting the composition comprising compound I.

In one embodiment, the present invention provides the intramuscular composition of Compound I, pharmaceutically acceptable salts or solvates thereof; and at least one pharmaceutically acceptable excipient; wherein the composition is able to deliver AUC₍₀₋ₜ₎ of the fulvestrant as compared to standard Fulvestrant injectable preparation in the ratio of 2:1 after injecting the composition comprising compound I, pharmaceutically acceptable salts or solvates thereof. In one embodiment the ratio of Cmax obtained from the composition comprising compound I in comparison to standard Fulvestrant injectable preparation can be selected from 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 after injecting the composition comprising compound I, pharmaceutically acceptable salts or solvates thereof. In one embodiment AUC (o-t) measured can be selected from AUC_{(0-7 days)}, AUC_{(0-14 days)}, AUC_{(0-21 days)}, AUC_{(0-28 days)}, AUC_{(0-60 days)}, AUC_{(0-180 days)}.

### Abbreviation

hr Hour
obs observed or observation
ng/ml nanogram per millilitre
h. ng/ml hour. Nanogram per millilitre
1/hr One per hour
mL/min/kg millilitres per minute per kilogram
L/Kg litre per kilogram
Cₘₐₓ Maximum Plasma Concentration
Tₘₐₓ Time to Maximum Plasma Concentration
AUC Area Under the Concentration-Time Curve
AUCₗₐₛₜ AUC to the Last Measurable Concentration
AUC_{inf_obs} AUC Extrapolated to Infinity observation AUC %_{exp.pol} AUC % extrapolated
Kₑₗ Elimination rate constant
T_{1/2} Terminal Elimination Half-Life
Cl/F_{obs} Observed apparent total plasma clearance
Vz/F_{obs} Observed apparent volume of distribution
MRTₗₐₛₜ Mean Residence time to the Last Measurable Concentration
MCT Medium chain triglycerides
PLGA-PEG-PLGA Poly (D,L-lactide-co-glycolide)-polyethylene glycol-poly (D,L-lactide-co-glycolide)
q.s. Quantity sufficient

### EXAMPLES

### Example 1: Composition of Compound I

| Ingredients | % w/v |
|---|---|
| Compound I | 11.3 |
| Benzyl alcohol | 10 |
| Alcohol | 10 |
| Benzyl benzoate | 15 |
| Castor Oil | q.s. |

### Procedure:

1) Fulvestrant was mixed with alcohol and benzyl alcohol in the amounts defined for the particular composition with stirring till the clear solution was obtained.
2) Benzyl benzoate was added in above solution and mixed well.
3) Volume was made up to 100% using castor oil and mixed well to get uniform solution.

### Example 2: Composition of Compound I

| Ingredients | % w/v |
|---|---|
| Compound I | 11.3 |
| Benzyl alcohol | 10 |
| Alcohol | 20 |
| Propylene glycol | 2.8 |
| Sorbitan monolaurate | 0.2 |
| Poloxamer 188 | 0.2 |
| Castor oil | q.s. |

### Procedure:

1) Compound I was mixed with alcohol and benzyl alcohol in the amounts defined for the particular composition with stirring till the clear solution was obtained.
2) Propylene glycol, sorbitan monolaurate, poloxamer 188 was added in the above solution and mixed well to get clear solution.
3) Volume was made up to 100% using castor oil and mixed well to get uniform solution.

### Example 3: Composition of Compound I

| Ingredients | % w/v |
|---|---|
| Compound I | 11.3 |
| Benzyl alcohol | 10 |
| Alcohol | 10 |
| Castor oil | 60 |
| MCT | q.s. |

### Procedure:

1) Compound I was mixed with alcohol and benzyl alcohol in the amounts defined for the particular composition with stirring till the clear solution was obtained.
2) Castor oil was added in the above solution and mixed well.
3) Volume was made up to 100% using MCT and mixed well to get uniform solution.

### Example 4: Composition of Compound I

| Ingredients | % w/v |
|---|---|
| Compound I | 11.3 |
| Benzyl alcohol | 10 |
| Alcohol | 10 |
| Polysorbat 80 | 0.12 |
| Alpha-Tocopherol | 0.06 |
| Castor oil | q.s. |

### Procedure:

1) Compound I was mixed with alcohol and benzyl alcohol in the amounts defined for the particular composition with stirring till the clear solution was obtained.
2) Polysorbat 80 and alpha tocopherol was added in above solution and mixed well.
3) Volume was made up to 100% using castor oil and mixed well to get uniform solution.

### Example 5: Composition of Compound I

| Ingredients | % w/v |
|---|---|
| Compound I | 11.3 |
| Benzyl alcohol | 10 |
| Propylene glycol | 10 |
| Castor oil | 60 |
| MCT | q.s. |

### Procedure:

1) Compound I was mixed with propylene glycol and benzyl alcohol in the amounts defined for the particular composition with stirring till the clear solution was obtained.
2) Castor oil was added in the above solution and mixed well.
3) Volume was made up to 100% using MCT and mixed well to get uniform solution.

### Example 6: Composition of Compound I

| Ingredients | % w/v |
|---|---|
| Compound I | 11.3 |
| Benzyl alcohol | 10 |
| Propylene glycol | 10 |
| Polysorbat 80 | 0.12 |
| Alpha-Tocopherol | 0.06 |
| Castor oil | 50 |
| MCT | q.s. |

### Procedure:

1) Compound I was mixed with propylene glycol and benzyl alcohol in the amounts defined for the particular composition with stirring till the clear solution was obtained
2) Polysorbat 80 and alpha tocopherol was added in above solution and mixed well.
3) Castor oil was added in the above solution and mixed well.
4) Volume was made up to 100% using MCT and mixed well to get uniform solution.

### Example 7: Composition of Compound I

| Ingredients | % W/V |
|---|---|
| Compound I | 10-25% |
| PLGA Poly (D,L-lactide-co-glycolide) | 1-50% |
| N-methylpyrrolidone | q.s. |

### Procedure:

1) Compound I was mixed with N-methylpyrrolidone in the amounts defined for the particular composition with stirring till the clear solution was obtained
2) PLGA was added in above solution and mixed well.
3) Volume was made up to 100% using N-methylpyrrolidone and mixed well to get uniform solution.

### Example 8: Composition of Compound I

| Ingredients | % W/V |
|---|---|
| Compound I | 10-25% |
| PLGA-PEG-PLGA block co-polymer | 1-50% |
| N-methylpyrrolidone | q.s. |

### Procedure:

1) Compound I was mixed with N-methylpyrrolidone in the amounts defined for the particular composition with stirring till the clear solution was obtained
2) PLGA-PEG-PLGA was added to block co-polymer in the above solution and mixed well.
3) Volume was made up to 100% using N-methylpyrrolidone and mixed well to get uniform solution.

### Example 9: Composition of Compound I

| Ingredients | % W/V |
|---|---|
| Compound I | 10-25% |
| PLGA | 1-50% |
| Benzyl alcohol | 5-10% |
| Alcohol | q.s. |

### Procedure:

1) Compound I was mixed with Alcohol in the amounts defined for the particular composition with stirring till the clear solution was obtained
2) PLGA was mixed with Benzyl alcohol in the amounts defined for the particular composition with stirring till the clear solution was obtained.
3) Mixed both the above solutions and volume was made up to 100% using Alcohol and mixed well to get uniform solution.

### Example 10: Composition of Compound I

| | | |
|---|---|---|
| Sr. No. | Ingredients | % W/V |

Microspheres

| | | |
|---|---|---|
| 1 | Compound I | 10-25% |
| 2 | PLGA | 1-50% |
| 3 | Organic solvent | q.s. |

Diluent

| | | |
|---|---|---|
| 4 | Carboxy methyl cellulose | 1-5% |
| 5 | Polysorbat 80 | 0.1-1% |
| 6 | Water for injection | q.s. |

### Procedure:

1) Compound I and PLGA was dissolved in organic solvent (oil phase). The oil phase was then dispersed in the 0.5% aqueous solution of polyvinyl alcohol to produce a O/W emulsion. The emulsion was stirred under nitrogen to evaporate organic solvent to solidify the oil phase. The microcapsules were collected by filtration, rinsed with water and dried. Microspheres stored as separate component in glass vial.
2) Carboxy methyl cellulose and Polysorbat 80 was dissolved in Water for injection. This solution filled in the syringe and used as diluents to resuspend the microsphere at the time of administration.

### Example 11: Composition of Compound I

| Ingredients | % W/V |
|---|---|
| Compound I | 10-25% |
| Poloxamer 188 | 0.05-2% |
| Benzyl alcohol | 5-10% |
| Propylene glycol | 5-10% |
| Alcohol | 10% |
| Castor oil | 10-70% |
| MCT | q.s. |

### Procedure:

1) Compound I was mixed with alcohol and benzyl alcohol in the amounts defined for the particular composition with stirring till the clear solution was obtained.
2) Propylene glycol, poloxamer 188 was added in above solution and mixed well to get clear solution.
3) Added castor oil in above solution and mixed well to get clear solution.
4) Volume was made up to 100% using MCT and mixed well to get uniform solution.

### Example 12: Composition of Compound I

| Ingredients | % W/V |
|---|---|
| Compound I | 10-25% |
| sucrose acetate isobutyrate | 10-70% |
| Benzyl alcohol | 10-20% |
| Alcohol | q.s. |

### Procedure:

1) Compound I was mixed with Alcohol in the amounts defined for the particular composition with stirring till the clear solution was obtained.
2) Sucrose acetate isobutyrate mixed with Benzyl alcohol in the amounts defined for the particular composition with stirring till the clear solution was obtained.
3) Mixed both the above solution and volume was made up to 100% using alcohol. The solution was further mixed well to tilla uniform solution was obtained.

### Example 13: Composition of Compound I

| Ingredients | % W/V |
|---|---|
| Compound I | 10-25% |
| sucrose acetate isobutyrate | 10-70% |
| Benzyl alcohol | 10-20% |
| DMA(N,N-Dimethyl acetamide) | q.s. |

### Procedure:

1) Compound I was mixed with DMA (N,N-Dimethyl acetamide) in the amounts defined for the particular composition with stirring till the clear solution was obtained.
2) Sucrose acetate isobutyrate mixed with Benzyl alcohol in the amounts defined for the particular composition with stirring till the clear solution was obtained.
3) Mixed both the above solution and volume was made up to 100% using alcohol. The solution was further mixed well to tilla uniform solution was obtained.

### Example 14: Composition of Compound I

| Ingredients | % W/V |
|---|---|
| Compound I | 10-25% |
| sucrose acetate isobutyrate | 10-70% |
| Benzyl alcohol | 10 |
| Alcohol | 10% |
| Benzyl benzoate | 15% |
| MCT | q.s. |

### Procedure:

1) Compound I was mixed with alcohol in the amounts defined for the particular composition with stirring till the clear solution was obtained.
2) Sucrose acetate isobutyrate was mixed with benzyl alcohol in the amounts defined for the particular composition with stirring till the clear solution was obtained.
3) Mix both the solution and add Benzyl benzoate and volume make up to 100% using MCT and mix well to get uniform solution.

### Example 15: Stability of Compound I in Solubilised Composition

Composition of compound I with 113 mg/ml concentration
(equivalent to 100 mg/ml Fulvestrant concentration)

| Ingredients | % w/v |
|---|---|
| Compound I | 11.3 |
| Benzyl alcohol | 10 |
| Alcohol | 10 |
| Benzyl benzoate | 15 |
| Castor Oil | q.s. |

| | Initial | Room Temp (45 Days) |
|---|---|---|
| Description | Clear colorless to light | Clear colorless to light |
| | yellow solution | yellow solution |
| Assay of Compound I | 104.0% | 103.3% |
| (%) | | |
| Visual Inspection | Clear solution, No | Clear solution, No |
| | precipitation observed | precipitation observed |

### Procedure:

1) Compound I was mixed with alcohol and benzyl alcohol in the amounts defined for the particular composition with stirring. Stirred the solution to get the clear solution.
2) Benzyl benzoate was added in above solution and mixed well.
3) Volume was made up to 100% using castor oil and mixed well to get uniform solution.

### Example 16: Stability of Compound I in Solubilised Composition

Composition with 169.5 mg/ml of compound I (equivalent
to 150 mg/ml Fulvestrant concentration)

| Ingredients | % w/v |
|---|---|
| Compound I | 16.95 |
| Benzyl alcohol | 10 |
| Alcohol | 10 |
| Benzyl benzoate | 15 |
| Castor Oil | q.s. |

| | Initial | Room Temp (30 Days) |
|---|---|---|
| Description | Clear colorless to light | Clear colorless to light |
| | yellow solution | yellow solution |
| Assay of compound I(%) | 100.4% | 95.30% |
| Visual Inspection | Clear solution, No | Clear solution, No |
| | precipitation observed | precipitation observed |

### Procedure:

1) Compound I was mixed with alcohol and benzyl alcohol in the amounts defined for the particular composition with stirring. Stirred the solution to get the clear solution.
2) Benzyl benzoate was added in above solution and mixed well.
3) Volume was made up to 100% using castor oil and mixed well to get uniform solution.

### Example 17: Stability of Compound I in Solubilised Composition

Composition with 226 mg/ml of compound I (equivalent
to 200 mg/ml Fulvestrant concentration)

| Ingredients | % w/v |
|---|---|
| Compound I | 22.6 |
| Benzyl alcohol | 10 |
| Alcohol | 10 |
| Benzyl benzoate | 15 |
| Castor Oil | q.s. |

| | Initial | Room Temp (30 Days) |
|---|---|---|
| Description | Clear colorless to | Clear colorless to light |
| | light yellow solution | yellow solution |
| Assay of compound I(%) | 101.8% | 98% |
| Visual Inspection | Clear solution, No | Clear solution, No |
| | precipitation observed | precipitation observed |

### Procedure:

1) Compound I was mixed with alcohol and benzyl alcohol in the amounts defined for the particular composition with stirring. Stirred the solution to get the clear solution.
2) Benzyl benzoate was added in above solution and mixed well.
3) Volume was made up to 100% using castor oil and mixed well to get uniform solution.

### Example 18: Pharmacokinetic Data in Rats

The objective of the study was to evaluate single site Vs. two site IM injection of compound I and it impact on pharmacokinetic exposure in female rats. Study was performed in female SD rats with n=6 animals per group. Faslodex 52 mg/kg (human equivalent dose) was administrated at two IM sites by dividing dose volume to half. In similar manner, 59 mg/kg (100 mg/mL compound I concentration) (52 mg/kg Fulvestrant equivalent) was administered as single injection (one IM site) or two IM injection by dividing dose volume in half. Blood was collected at time points 0.25, 0.50, 1, 2, 4, 8, 12, 24, 48, 72, 96, 120, 144, 168 hrs. At each time point, −0.3 mL blood was collected in tubes containing Li-heparin as anti-coagulant. For plasma separation, blood was centrifuges at 5000 rpm for 10 minutes at 4° C. and collected plasma was stored at −80° C. until further analysis.

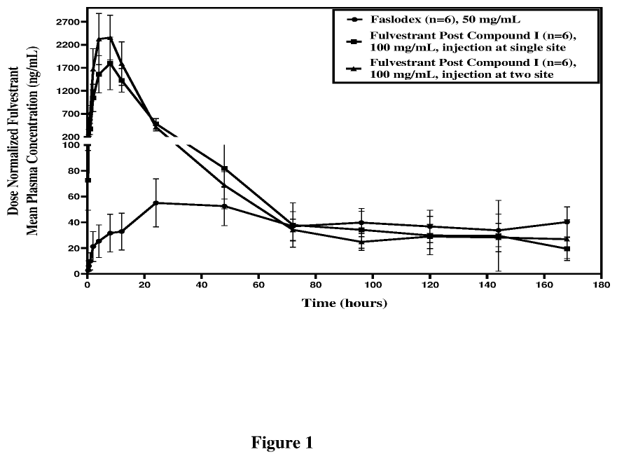

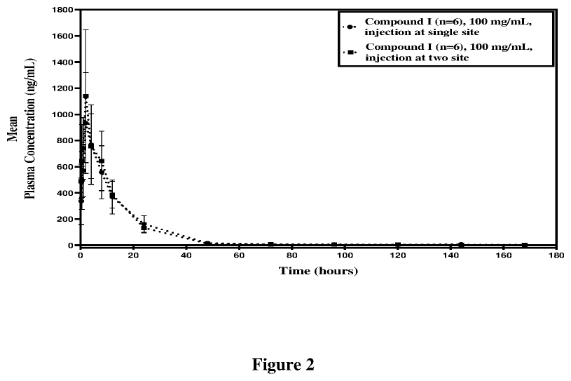

Bioanalysis in plasma samples was performed using fit-for-purpose LC-MS/MS method for the quantification Fulvestrant and Compound I. Linearity range for Fulvestrant and Compound I analytes were 2 to 2000 ng/mL. The bioanalytical summary for Fulvestrant concentration in all three groups is shown in table 1 and graphical representation is shown in FIG. 1 Concentration of Compound I is shown in table 2 and graphical representation is shown in FIG. 2. The plasma pharmacokinetic parameters for Fulvestrant and Compound I were calculated using standard non-compartmental analysis (Phoenix® software, version 8.1, Pharsight Corporation, Mountain View, California 94040/USA) linear trapezoidal method with linear interpolation.

TABLE 1
Arithmetic Mean ± SD plasma pharmacokinetic parameters of fulvestrant
following a single intramuscular administration to Female SD rats

| | | Group 2: | Group 3: |
|---|---|---|---|
| | | Fulvestrant from | Fulvestrant from |
| | | Compound I | Compound I |
| | | −100 mg/mL | −100 mg/mL |
| | Group 1: | (59 mg/kg), | (59 mg/kg), |
| | Faslodex ® | IM injection at | IM injection at |
| PK parameters | (52 mg/kg) | single site | two sites |
| Cₘₐₓ (ng/mL) | 58.03 ± 16.6 | 1907.49 ± 503.49 | 2459.29 ± 457.82 |
| Tₘₐₓ (hr) | 40.00 ± 19.60 | 6.00 ± 2.19 | 8.00 ± 2.53 |
| AUCₗₐₛₜ (h · ng/mL) | 6747.18 ± 1993.76 | 36723.82 ± 7171.21 | 42705.80 ± 6084.84 |

Note:
Values are expressed as Mean ± SD.

TABLE 2
Arithmetic Mean ± SD plasma pharmacokinetic
parameters of Compound I following a single
intramuscular administration to Female SD Rats

| | Group 2: | Group 3: |
|---|---|---|
| | Compound I | Compound I |
| | −100 mg/mL | −100 mg/mL |
| | (59 mg/kg), | (59 mg/kg), |
| | IM injection at | IM injection at |
| PK parameters | single site | two sites |
| Cₘₐₓ (ng/mL) | 968.70 ± 349.1 | 1145.26 ± 500.12 |
| Tₘₐₓ (h) | 3.17 ± 2.56 | 1.83 ± 0.41 |
| AUCₗₐₛₜ (h · ng/mL) | 12407.64 ± 3425.35 | 12765.13 ± 3116.77 |

Note:
Values are expressed as Mean ± SD.

### Example 19: Pharmacokinetic Data in Dog

### Procedure:

A total of 9 female Beagle dogs were used in this study. The study was performed as a parallel design study.

On the day of dosing, each dog was identified by animal ID. Each kennel, housing a single dog was identified by cage card listing cage number, animal identification number, sex, study number, in-life experiment staffs and end dates.

All dogs were administered intramuscularly at the gluteal region.

Dose volume was equally divided and administered to two IM sites.

Faslodex (Fulvestrant and Compound I were dosed as shown in table 3

TABLE 3
Group allocation and treatment

| | | | Dose | Formulation | |
|---|---|---|---|---|---|
| | Treatment | Animal | Volume | strength | |
| Group | (Dose: mg/kg) | ID No. | (mL/kg) | (mg/mL) | ROA |
| 1 | Faslodex ® | 1, 2 & 3 | 0.3 | 50 | IM |
| | (15 mg/kg) | | | | |
| 2 | Compound I | 4, 5 & 6 | 0.34 | 50 | |
| | (50 mg/mL) | | | | |
| | (17 mg/kg | | | | |
| | (equivalent to. 15 | | | | |
| | mg/kg Fulvestrant) | | | | |
| 3 | Compound I | 7, 8 & 9 | 0.17 | 100 | |
| | (100 mg/mL) | | | | |
| | (17 mg/kg | | | | |
| | (equivalent to. 15 | | | | |
| | mg/kg Fulvestrant) | | | | |

Blood samples were collected through jugular vein at 0 (Pre-dose), 0.25, 0.5, 1, 2, 4, 8, 12, 24, 48, 72, 96, 120, 144, 168, 192, 216, 240, 264, 288, 312, and 336 hours post-dose (Total 22 time points/dog). At each time point, −0.5 mL of blood was withdrawn and transferred into a pre-labelled K₂EDTA coated vacutainer tubes and mixed gently by inverting the tube to facilitate mixing of anticoagulant with the blood. Blood samples were kept on gel packs until centrifugation. The collected blood samples were centrifuged at 5000 rpm for 5 min at 4° C. Plasma was separated after centrifugation. All plasma samples were transferred into pre-labeled (Animal ID No., Time point, Study No., and Group) tubes and stored at −70±10° C. until analysis.

Bioanalysis in plasma samples was performed using fit-for-purpose LC-MS/MS method for the quantification of—Group 1—Fulvestrant
Group 2—Fulvestrant and Compound I
Group 3—Fulvestrant and Compound I

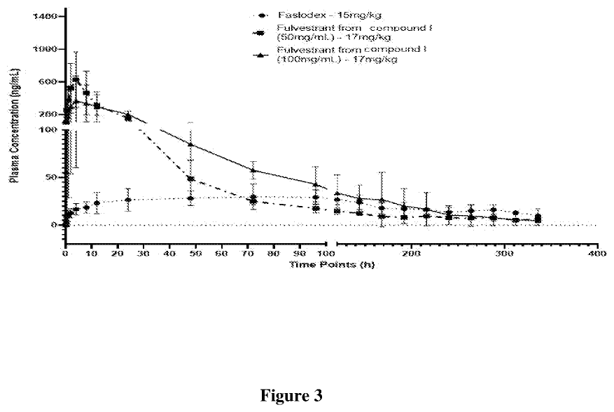

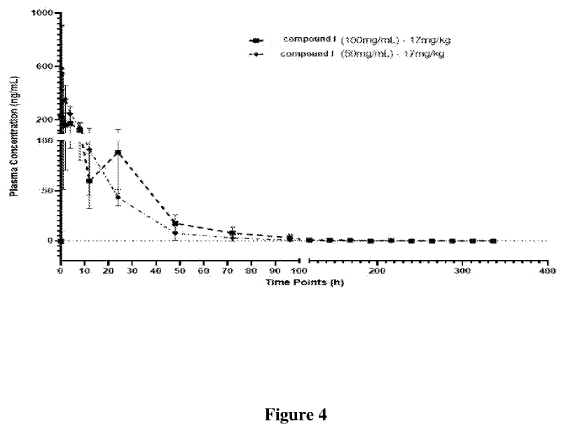

Linearity range for Fulvestrant and Compound I analytes were 1 to 1000 ng/mL. The bioanalytical summary for Fulvestrant concentration in all three groups is shown in table 4 and graphical representation is shown in FIG. 3 Concentration of pro-drug Compound I is shown in table 5 and graphical representation is shown in FIG. 4. The plasma pharmacokinetic parameters for Fulvestrant and Compound I were calculated using standard non-compartmental analysis (Phoenix® software, version 8.3, Pharsight Corporation, Mountain View, California 94040/USA) linear trapezoidal method with linear interpolation. Ratio of fulvestrant obtained after dosing Compound I to that of Faslodex was calculated for parameters Cmax and AUC is reported in Table 6(A) and Table 6(B).

TABLE 4
Arithmetic Mean ± SD plasma pharmacokinetic
parameters of fulvestrant following a single
intramuscular administration to Female Beagle dogs

| | | Group 2: | Group 3: |
|---|---|---|---|
| | | Fulvestrant from | Fulvestrant from |
| | Group 1: | Compound I | Compound I |
| | Faslodex ® | −50 mg/mL | −100 mg/mL |
| PK parameters | (15 mg/kg) | (17 mg/kg) | (17 mg/kg) |
| Cₘₐₓ (ng/mL) | 37.5 ± 7.91 | 628 ± 342 | 420 ± 241 |
| Tₘₐₓ (hr) | 80 ± 13.86 | 4 | 12 ± 10.58 |
| AUCₗₐₛₜ (h · | 7000 ± 271 | 14500 ± 3520 | 17400 ± 420 |
| ng/mL) | | | |
| AUC_{inf}_{_{—}}_{obs} | 8950 ± 464 | 15500 ± 3340 | 17900 ± 624 |
| (h · ng/mL) | | | |
| AUC_{% exp. pol} (%) | 20.6 ± 0.948 | 6.9 ± 2.56 | 2.5 ± 2.86 |

Note:
Values are expressed as Mean ± SD.

TABLE 5
Arithmetic Mean ± SD plasma pharmacokinetic
parameters of Compound I following a single intramuscular
administration to Female Beagle dogs

| | Group 2: | Group 3: |
|---|---|---|
| | Compound I | Compound I |
| | −50 mg/mL | −100 mg/mL |
| PK parameters | (17 mg/kg) | (17 mg/kg) |
| Cₘₐₓ (ng/mL) | 624 ± 274 | 272 ± 183 |
| Tₘₐₓ (h) | 0.83 ± 1.01 | 1.83 ± 1.89 |
| AUCₗₐₛₜ (h · ng/mL) | 4220 ± 1060 | 4320 ± 1410 |
| AUC_{inf}_{_{—}}_{obs} (h · ng/mL) | 4260 ± 1060 | 4360 ± 1390 |
| AUC_{% exp. pol} (%) | 0.967 ± 0.255 | 0.974 ± 0.886 |

Note:
Values are expressed as Mean ± SD.

TABLE 6(A)
Ratio comparison of Cmax and AUC of Compound I with Faslodex

| | Cmax ratio | AUC0-14 ratio |
|---|---|---|
| Group | to Faslodex | to Faslodex |
| Faslodex | 1 | 1 |
| Fulvestrant from Compound I | 16.7 | 2.07 |
| (50 mg/ml) | | |
| Fulvestrant from Compound I | 11.2 | 2.49 |
| (100 mg/ml) | | |

TABLE 6(B)
% Cmax and AUC Comparison of Compound I with Faslodex

| | % Cmax | % AUC0-14 |
|---|---|---|
| | compared | compared |
| Group | to Faslodex | Faslodex |
| Faslodex | 100 | 100 |
| Fulvestrant from Compound I | 1670 | 207 |
| (50 mg/ml) | | |
| Fulvestrant from Compound I | 1120 | 249 |
| (100 mg/ml) | | |

| Ingredients | % w/v |
|---|---|
| Compound I | 16.9 |
| Alcohol | 20 |
| Benzyl benzoate | 15 |
| Castor oil | q.s. |

Procedure: 1) Compound I was mixed with alcohol in the amounts defined for the particular composition with stirring till the clear solution was obtained
2) Benzyl benzoate was added in above solution and mixed well.
3) Volume was made up to 100% using Castor oil and mixed well to get uniform solution.

### Example 21: Composition of Compound I

| Ingredients | % w/v |
|---|---|
| Compound I | 16.9 |
| Benzyl alcohol | 5 |
| Alcohol | 15 |
| Benzyl benzoate | 15 |
| Castor oil | q.s. |

Procedure: 1) Compound I was mixed with alcohol and, benzyl alcohol in the amounts defined for the particular composition with stirring till the clear solution was obtained
2) Benzyl benzoate was added in above solution and mixed well.
3) Volume was made up to 100% using Castor oil and mixed well to get uniform solution.

### Example 22: Composition of Compound I

| Ingredients | % w/w |
|---|---|
| Compound I | 6.67 |
| Dimethylacetamide | 66.67 |
| PLGA | 26.67 |

Procedure: 1) Compound I was mixed with dimethylacetamide in the amounts defined for the particular composition with stirring till the clear solution was obtained
2) PLGA was added in above solution and mixed well to get uniform solution.

### Example 23: Composition of Compound I

| Ingredients | % w/v |
|---|---|
| Compound I | 5-30 |
| Alpha-lipoic acid | 5-50 |
| Benzyl alcohol | 10 |
| Alcohol | 10 |
| Dimethylacetamide | 5-20 |
| Benzyl benzoate | q.s. |

Procedure: 1) Compound I was mixed with alcohol, benzyl alcohol and dimethylacetamide in the amounts defined for the particular composition with stirring till the clear solution was obtained
2) Alpha-lipoic acid was added in above solution and mixed well.
3) Volume was made up to 100% using Benzyl benzoate and mixed well to get uniform solution.

### Example 24: Pharmacokinetic Study in Female Beagle Dog

### Procedure:

A total of 2 female beagle dogs were used in this study.

On the day of dosing, each dog was identified by animal ID. Each kennel, housing a single dog was identified by cage card listing cage number, animal identification number, sex, study number, in-life experiment starts and end dates.

All dogs were administered intramuscularly at the gluteal region.

Dose volume was equally divided and administered to two IM sites.

Compound I was dosed as shown in table 7

TABLE 7
Group allocation and treatment

| | | | Dose | Formulation | |
|---|---|---|---|---|---|
| | Treatment | Animal | Volume | strength | |
| Group | (Dose: mg/kg) | ID No. | (mL/kg) | (mg/mL) | ROA |
| 1 | Compound I | 1, 2 | 0.1 | 169.5 | IM |
| | (169.5 mg/mL) | | | | |
| | (17 mg/kg, equivalent | | | | |
| | to 15 mg/kg | | | | |
| | fulvestrant) | | | | |

Blood samples were collected through jugular vein at 0 (Pre-dose), 0.25, 0.5, 1, 2, 4, 8, 12, 24, 48, 72, 96, 120, 144, 168, 192, 216, 240, 264, 288, 312, and 366 hours post-dose (Total 22 time points/dog). At each time point, −0.5 mL of blood was withdrawn and transferred into a pre-labeled K₂EDTA coated vacutainer tubes and mixed gently by inverting the tube to facilitate mixing of anticoagulant with the blood. Blood samples were kept on gel packs until centrifugation. The collected blood samples were centrifuged at 5000 rpm for 5 min at 4° C. Plasma was separated after centrifugation. All plasma samples were transferred into pre-labeled (Animal ID No., Time point, Study No., and Group) tubes and stored at −70±10° C. until analysis.

Bioanalysis of plasma samples was performed using fit-for-purpose LC-MS/MS method for the quantification of fulvestrant and Compound I.

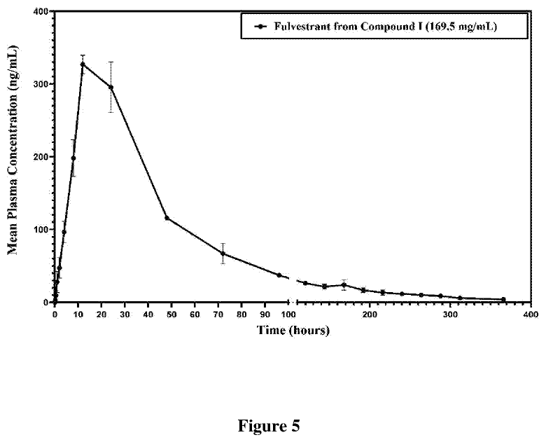

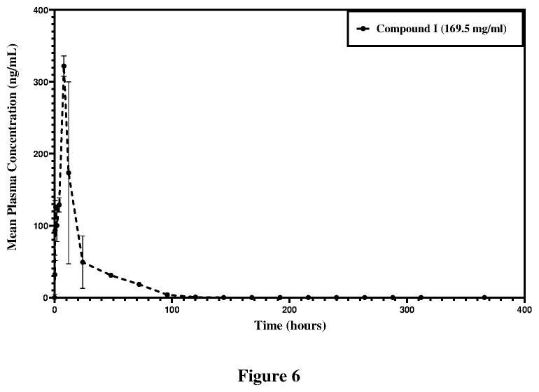

Linearity range for Fulvestrant and Compound I analytes were 1 to 1000 ng/mL. The bioanalytical summary for fulvestrant concentration is shown in table 8 and graphical representation is shown in FIG. 5. Concentration of Compound I is shown in table 9 and graphical representation is shown in FIG. 6. The plasmapharmacokinetic parameters for Fulvestrant and Compound I were calculated using standard non-compartmental analysis (Phoenix® software, version 8.3, Pharsight Corporation, Mountain View, California 94040/USA) linear trapezoidal method with linear interpolation.

TABLE 8
Arithmetic Mean ± SD plasma pharmacokinetic
parameters of fulvestrant following a single
intramuscular administration to Female Beagle dogs

| | Fulvestrant from Compound I |
|---|---|
| | −169.5 mg/mL |
| PK parameters | (17 mg/kg) |
| Cₘₐₓ (ng/mL) | 328 ± 11.3 |
| Tₘₐₓ (hr) | 12-24 |
| AUCₗₐₛₜ (h · ng/mL) | 17900 ± 185 |
| AUC_{inf}_{_{—}}_{obs} (h · ng/mL) | 18300 ± 300 |
| AUC_{% exp. pol} (%) | 2.14 ± 0.595 |

Note:
Values are expressed as Mean ± SD.

TABLE 9
Arithmetic Mean ± SD plasma pharmacokinetic
parameters of Compound I following a single intramuscular
administration to Female Beagle dogs

| | Compound I |
|---|---|
| | −169.5 mg/mL |
| PK parameters | (17 mg/kg) |
| Cₘₐₓ (ng/mL) | 322 ± 14.1 |
| Tₘₐₓ (h) | 8 |
| AUCₗₐₛₜ (h · ng/mL) | 5510 ± 1880 |
| AUC_{inf}_{_{—}}_{obs} (h · ng/mL) | 5540 ± 1880 |
| AUC_{% exp. pol} (%) | 0.578 ± 0.303 |

Note:
Values are expressed as Mean ± SD.

### Example 25: Pharmacokinetic Study of Compound I in Male Cynomolgus Monkey

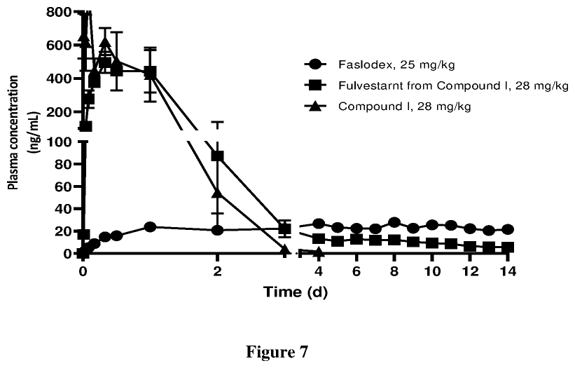

Procedure: In this single-dose pharmacokinetics study, four Non-Naïve male Cyno monkeys (Age—2.5-6 years, Weight 2-5 kg) were randomly assigned into two groups to evaluate the pharmacokinetics of Faslodex (1 animal) Vs. Compound I (3 animals) following single intramuscular (IM) administration. The test articles were monitored in plasma for up to 336 h (14 days) following dosing. Faslodex at dose of 25 mg/kg (50 mg/mL fulvestrant strength, 500 mg human equivalent dose) was intramuscularly administrated into monkey at study Day 0. The dosage volume 0.5 mL/kg was equally divided and administered at two sites. Compound I at dose 28.3 mg/kg (25 mg/kg Fulvestrant equivalent) having strength 169.5 mg/mL (150 mg/mL fulvestrant equivalent) was intramuscularly administrated into monkeys at study Day 0. The dosage volume of 0.15 mL/kg was administered on one IM site. Blood samples were collected through cephalic or saphenous vein at 0 (Pre-dose), 0.25, 1, 2, 4, 8, 12, 24, 48, 72, 96, 120, 144, 168, 192, 216, 240, 264, 288, 312, and 336 hours. Plasma concentrations of Compound I and fulvestrant were determined using LC/MS/MS to construct semi-logarithmic plasma concentration-time curves. Pharmacokinetic analysis was performed using non-compartmental model. Pharmacokinetic profile is shown in FIG. 7 and pharmacokinetic parameters are mentioned in Table 10

TABLE 10
Pharmacokinetic parameters after administration of
Faslodex or Compound I in male cynomolgus monkeys

| | Group 1: | Group 2: | Group 2: Fulvestrant |
|---|---|---|---|
| Parameter | Faslodex | Compound I | released from Compound I |
| C_max (ng/mL) | 27.89 | 1081 | 533 |
| AUC_0- | 7656 | 17460 | 18915 |
| t(h*ng/mL) | | | |
| T_½(h) | 304.49 | 12.79 | 138.26 |
| T_max(h) | 192.00 | 3.42 | 18.67 |
| Tlast (h) | 336.00 | 152.00 | 336.00 |

1. An intramuscular composition comprising compound I, 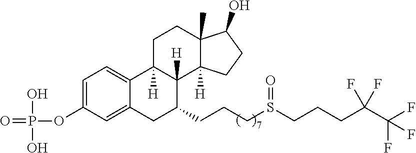
pharmaceutically acceptable salts or solvates thereof; and at least one pharmaceutically acceptable excipient, wherein the composition administered to a subject in need thereof has concentration of compound I, pharmaceutically acceptable salts or solvates thereof from about 10 mg/ml to about 300 mg/ml.

2. The composition according to claim 1, wherein the concentration of compound I, pharmaceutically acceptable salts or solvates thereof; is selected from group consisting of about 20 mg/ml to about 300 mg/ml, about 30 mg/ml to about 300 mg/ml, about 40 mg/ml to about 300 mg/ml, about 50 mg/ml to about 300 mg/ml, about 60 mg/ml to about 300 mg/ml, about 70 mg/ml to about 300 mg/ml, about 80 mg/ml to about 300 mg/ml, about 90 mg/ml to about 300 mg/ml, about 100 mg/ml to about 300 mg/ml, about 110 mg/ml to about 300 mg/ml, about 120 mg/ml to about 300 mg/ml, about 130 mg/ml to about 300 mg/ml, about 140 mg/ml to about 300 mg/ml, about 150 mg/ml to about 300 mg/ml, about 160 mg/ml to about 300 mg/ml, about 170 mg/ml to about 300 mg/ml, about 180 mg/ml to about 300 mg/ml, about 190 mg/ml to about 300 mg/ml, about 200 mg/ml to about 300 mg/ml, about 210 mg/ml to about 300 mg/ml, about 220 mg/ml to about 300 mg/ml, about 230 mg/ml to about 300 mg/ml, about 240 mg/ml to about 300 mg/ml, about 250 mg/ml to about 300 mg/ml, about 260 mg/ml to about 300 mg/ml, about 270 mg/ml to about 300 mg/ml, about 280 mg/ml to about 300 mg/ml, or about 290 mg/ml to about 300 mg/ml.

3. The composition according to claim 1, wherein the concentration of compound I or pharmaceutically acceptable salts and solvates thereof; is selected from about 20 mg/ml, about 30 mg/ml, about 40 mg/ml, about 50 mg/ml, about 60 mg/ml, about 70, mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml, about 260 mg/ml, about 270 mg/ml, about 280 mg/ml, about 290 mg/ml, or about 300 mg/mi.

4. An intramuscular composition comprising compound I, 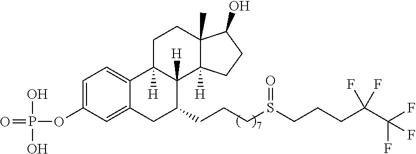
pharmaceutically acceptable salts or solvates thereof; and at least one pharmaceutically acceptable excipient, wherein the composition administered to a subject in need thereof maintains blood plasma fulvestrant concentration for a desired period of time after injecting the composition comprising compound I, pharmaceutically acceptable salts or solvates thereof.

5. The composition according to claim 4, wherein the desired period of time to maintain blood plasma fulvestrant concentration is selected from at least one week, at least two weeks, at least three weeks, at least four weeks, at least one month, at least two months or at least three months.

6. The composition according to claim 4, wherein the composition administered to a subject in need thereof maintains blood plasma fulvestrant concentration of at least 1 ng/ml for a desired period of time after injecting the composition comprising compound I, pharmaceutically acceptable salts or solvates thereof.

7. The composition according to claim 6, wherein the composition administered to a subject in need thereof maintains blood plasma fulvestrant concentration selected from group consisting of at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml, at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48 ng/ml, at least 49 ng/ml, at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml.

8. The composition according to claim 4, wherein the composition administered to a subject in need thereof maintains blood plasma fulvestrant concentration of about 1 ng/ml to about 500 ng/ml for a desired period of time after injecting the composition comprising compound I, pharmaceutically acceptable salts or solvates thereof.

9. The composition according to claim 8, wherein the composition administered to a subject in need thereof maintains blood plasma fulvestrant concentration selected from group consisting of about 1 ng/ml to about 490 ng/ml, about 1 ng/ml to about 480 ng/ml, about 1 ng/ml to about 470 ng/ml, about 1 ng/ml to about 460 ng/ml, about 1 ng/ml to about 450 ng/ml, about 1 ng/ml to about 440 ng/ml, about 1 ng/ml to about 430 ng/ml, about 1 ng/ml to about 420 ng/ml, about 1 ng/ml to about 410 ng/ml, about 1 ng/ml to about 400 ng/ml, about 1 ng/ml to about 390 ng/ml, about 1 ng/ml to about 380 ng/ml, about 1 ng/ml to about 370 ng/ml, about 1 ng/ml to about 360 ng/ml, about 1 ng/ml to about 350 ng/ml, about 1 ng/ml to about 340 ng/ml, about 1 ng/ml to about 330 ng/ml, about 1 ng/ml to about 320 ng/ml, 1 ng/ml to about 310 ng/ml, about 1 ng/ml to about 300 ng/ml, about 1 ng/ml to about 290 ng/ml, about 1 ng/ml to about 280 ng/ml, about 1 ng/ml to about 270 ng/ml, about 1 ng/ml to about 260 ng/ml, about 1 ng/ml to about 250 ng/ml, about 1 ng/ml to about 240 ng/ml, about 1 ng/ml to about 230 ng/ml, about 1 ng/ml to about 220 ng/ml, about 1 ng/ml to about 210 ng/ml, about 1 ng/ml to about 200 ng/ml, about 1 ng/ml to about 190 ng/ml, about 1 ng/ml to about 180 ng/ml, about 1 ng/ml to about 170 ng/ml, about 1 ng/ml to about 160 ng/ml, about 1 ng/ml to about 150 ng/ml, about 1 ng/ml to about 140 ng/ml, about 1 ng/ml to about 130 ng/ml, about 1 ng/ml to about 120 ng/ml, about 1 ng/ml to about 110 ng/ml, or about 1 ng/ml to about 100 ng/ml.

10. The composition according to claim 9, wherein the composition administered to a subject in need thereof maintains blood plasma fulvestrant concentration selected from group consisting of about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml.

11. An intramuscular composition comprising compound I, 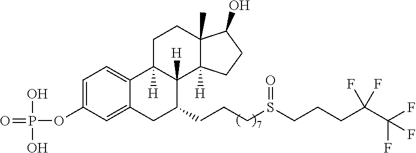
pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for desired period of time after injecting the composition comprising compound I, pharmaceutically acceptable salts or solvates thereof.

12. The composition according to claim 11, wherein the desired period of time is selected from group consisting of at least one week, at least two weeks, at least three weeks, at least four weeks, at least one month, at least two months or at least three months.

13. An intramuscular composition comprising compound I, 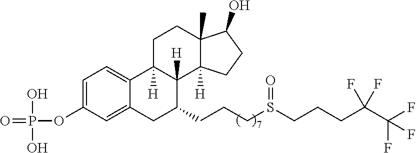
pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 500 ng/ml for desired period of time after injecting the composition comprising compound I, pharmaceutically acceptable salts or solvates thereof.

14. The composition according to claim 13, wherein the desired period of time is selected from group consisting of at least one week, at least two weeks, at least three weeks, at least four weeks, at least one month, at least two months or at least three months.

15. The composition according to claim 1, wherein at least one excipient is selected from oil, solvent, surfactant, antioxidant, polymer, or mixture thereof.

16. A method for treating a hormonal dependent benign or malignant disease of the breast or reproductive tract, comprising administering intramuscularly to a subject in need of such treatment the composition having compound I 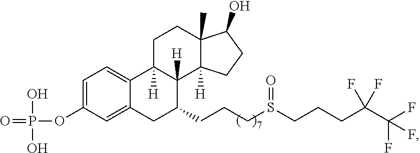
pharmaceutically acceptable salts or solvates thereof; and at least one pharmaceutically acceptable excipient, wherein the composition administered to a subject in need thereof has concentration of compound I, pharmaceutically acceptable salts or solvates thereof from about 10 mg/ml to about 300 mg/ml.

17. The method of treatment according to claim 16, wherein the concentration of compound I, pharmaceutically acceptable salts or solvates thereof; is selected from group consisting of about 20 mg/ml to about 300 mg/ml, about 30 mg/ml to about 300 mg/ml, about 40 mg/ml to about 300 mg/ml, about 50 mg/ml to about 300 mg/ml, about 60 mg/ml to about 300 mg/ml, about 70 mg/ml to about 300 mg/ml, about 80 mg/ml to about 300 mg/ml, about 90 mg/ml to about 300 mg/ml, about 100 mg/ml to about 300 mg/ml, about 110 mg/ml to about 300 mg/ml, about 120 mg/ml to about 300 mg/ml, about 130 mg/ml to about 300 mg/ml, about 140 mg/ml to about 300 mg/ml, about 150 mg/ml to about 300 mg/ml, about 160 mg/ml to about 300 mg/ml, about 170 mg/ml to about 300 mg/ml, about 180 mg/ml to about 300 mg/ml, about 190 mg/ml to about 300 mg/ml, about 200 mg/ml to about 300 mg/ml, about 210 mg/ml to about 300 mg/ml, about 220 mg/ml to about 300 mg/ml, about 230 mg/ml to about 300 mg/ml, about 240 mg/ml to about 300 mg/ml, about 250 mg/ml to about 300 mg/ml, about 260 mg/ml to about 300 mg/ml, about 270 mg/ml to about 300 mg/ml, about 280 mg/ml to about 300 mg/ml, or about 290 mg/ml to about 300 mg/ml.

18. The method of treatment according to claim 17, wherein the composition administered to a subject in need thereof has concentration of compound I, pharmaceutically acceptable salts or solvates thereof selected from about 20 mg/ml, about 30 mg/ml, about 40 mg/ml, about 50 mg/ml, about 60 mg/ml, about 70, mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 210 mg/ml, about 220 mg/ml, about 230 mg/ml, about 240 mg/ml, about 250 mg/ml, about 260 mg/ml, about 270 mg/ml, about 280 mg/ml, about 290 mg/ml or about 300 mg/ml.

19. A method for treating a hormonal dependent benign or malignant disease of the breast or reproductive tract comprising administering intramuscularly to a subject in need the intramuscular composition comprising compound I, 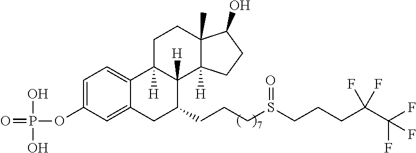
pharmaceutically acceptable salts or solvates thereof; and at least one pharmaceutically acceptable excipient, wherein the composition administered to a subject in need thereof maintains blood plasma fulvestrant concentration for a desired period of time after injecting the composition comprising compound I, pharmaceutically acceptable salts or solvates thereof

20. The method of treatment according to claim 19, wherein the composition administered to a subject in need thereof maintains blood plasma fulvestrant concentration of at least 1 ng/ml for desired period of time after injecting the composition comprising compound I, pharmaceutically acceptable salts or solvates thereof.

21. The method of treatment according to claim 19, wherein the desired period of time is selected from group consisting of at least one week, at least two weeks, at least three weeks, at least four weeks, at least one month, at least two months or at least three months.

22. The method of treatment according to claim 20, wherein the composition administered to a subject in need thereof maintains blood plasma fulvestrant concentration selected from the group consisting of at least 2 ng/ml, at least 3 ng/ml, at least 4 ng/ml, at least 5 ng/ml, at least 6 ng/ml, at least 7 ng/ml, at least 8 ng/ml, at least 9 ng/ml, at least 10 ng/ml, at least 11 ng/ml, at least 12 ng/ml, at least 13 ng/ml, at least 14 ng/ml, at least 15 ng/ml, at least 16 ng/ml, at least 17 ng/ml, at least 18 ng/ml, at least 19 ng/ml, at least 20 ng/ml, at least 21 ng/ml, at least 22 ng/ml, at least 23 ng/ml, at least 24 ng/ml, at least 25 ng/ml, at least 26 ng/ml, at least 27 ng/ml, at least 28 ng/ml, at least 29 ng/ml, at least 30 ng/ml, at least 31 ng/ml, at least 32 ng/ml, at least 33 ng/ml, at least 34 ng/ml, at least 35 ng/ml, at least 36 ng/ml, at least 37 ng/ml, at least 38 ng/ml, at least 39 ng/ml, at least 40 ng/ml, at least 41 ng/ml, at least 42 ng/ml, at least 43 ng/ml, at least 44 ng/ml, at least 45 ng/ml, at least 46 ng/ml, at least 47 ng/ml, at least 48 ng/ml, at least 49 ng/ml, at least 50 ng/ml, at least 51 ng/ml, at least 52 ng/ml, at least 53 ng/ml, at least 54 ng/ml, at least 55 ng/ml, at least 56 ng/ml, at least 57 ng/ml, at least 58 ng/ml, at least 59 ng/ml, at least 60 ng/ml, at least 61 ng/ml, at least 62 ng/ml, at least 63 ng/ml, at least 64 ng/ml, at least 65 ng/ml, at least 66 ng/ml, at least 67 ng/ml, at least 68 ng/ml, at least 69 ng/ml, at least 70 ng/ml, at least 71 ng/ml, at least 72 ng/ml, at least 73 ng/ml, at least 74 ng/ml, at least 75 ng/ml, at least 76 ng/ml, at least 77 ng/ml, at least 78 ng/ml, at least 79 ng/ml or at least 80 ng/ml, at least 81 ng/ml, at least 82 ng/ml, at least 83 ng/ml, at least 84 ng/ml, at least 85 ng/ml, at least 86 ng/ml, at least 87 ng/ml, at least 88 ng/ml, at least 89 ng/ml, at least 90 ng/ml, at least 91 ng/ml, at least 92 ng/ml, at least 93 ng/ml, at least 94 ng/ml, at least 95 ng/ml, at least 96 ng/ml, at least 97 ng/ml, at least 98, at least 99 ng/ml or at least 100 ng/ml.

23. The method of treatment according to claim 19, wherein the composition administered to a subject in need thereof maintains blood plasma fulvestrant concentration of about 1 ng/ml to about 500 ng/ml for desired period of time after injecting the composition comprising compound I, pharmaceutically acceptable salts or solvates thereof.

24. The method of treatment according to claim 23, wherein the composition administered to a subject in need thereof maintains blood plasma fulvestrant concentration selected from the group consisting of about 1 ng/ml to about 490 ng/ml, about 1 ng/ml to about 480 ng/ml, about 1 ng/ml to about 470 ng/ml, about 1 ng/ml to about 460 ng/ml, about 1 ng/ml to about 450 ng/ml, about 1 ng/ml to about 440 ng/ml, about 1 ng/ml to about 430 ng/ml, about 1 ng/ml to about 420 ng/ml, about 1 ng/ml to about 410 ng/ml, about 1 ng/ml to about 400 ng/ml, about 1 ng/ml to about 390 ng/ml, about 1 ng/ml to about 380 ng/ml, about 1 ng/ml to about 370 ng/ml, about 1 ng/ml to about 360 ng/ml, about 1 ng/ml to about 350 ng/ml, about 1 ng/ml to about 340 ng/ml, about 1 ng/ml to about 330 ng/ml, about 1 ng/ml to about 320 ng/ml, about 1 ng/ml to about 310 ng/ml, about 1 ng/ml to about 300 ng/ml, about 1 ng/ml to about 290 ng/ml, about 1 ng/ml to about 280 ng/ml, about 1 ng/ml to about 270 ng/ml, about 1 ng/ml to about 260 ng/ml, about 1 ng/ml to about 250 ng/ml, about 1 ng/ml to about 240 ng/ml, about 1 ng/ml to about 230 ng/ml, about 1 ng/ml to about 220 ng/ml, about 1 ng/ml to about 210 ng/ml, about 1 ng/ml to about 200 ng/ml, about 1 ng/ml to about 190 ng/ml, about 1 ng/ml to about 180 ng/ml, about 1 ng/ml to about 170 ng/ml, about 1 ng/ml to about 160 ng/ml, about 1 ng/ml to about 150 ng/ml, about 1 ng/ml to about 140 ng/ml, about 1 ng/ml to about 130 ng/ml, about 1 ng/ml to about 120 ng/ml, about 1 ng/ml to about 110 ng/ml, or about 1 ng/ml to about 100 ng/ml.

25. The method of treatment according to claim 23, wherein the composition administered to a subject in need thereof maintains blood plasma fulvestrant concentration selected from the group consisting of about 2 ng/ml to about 100 ng/ml, about 4 ng/ml to about 100 ng/ml, about 6 ng/ml to about 100 ng/ml, about 8 ng/ml to about 100 ng/ml, about 10 ng/ml to about 100 ng/ml, about 12 ng/ml to about 100 ng/ml, about 14 ng/ml to about 100 ng/ml, about 16 ng/ml to about 100 ng/ml, about 18 ng/ml to about 100 ng/ml, about 20 ng/ml to about 100 ng/ml, about 22 ng/ml to about 100 ng/ml, about 24 ng/ml to about 100 ng/ml, about 26 ng/ml to about 100 ng/ml, about 28 ng/ml to about 100 ng/ml or about 30 ng/ml to about 100 ng/ml after injecting the composition comprising compound I, pharmaceutically acceptable salts or solvates thereof.

26. A method for treating a hormonal dependent benign or malignant disease of the breast or reproductive tract comprising administering intramuscularly to a subject in need the intramuscular composition comprising compound I, 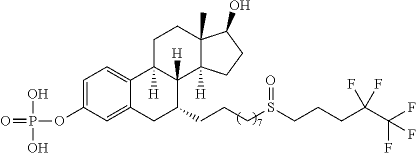
pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of at least 1 ng/ml for desired period of time after injecting the composition comprising compound I, pharmaceutically acceptable salts or solvates thereof.

27. The composition according to claim 26, wherein the desired period of time is selected from group consisting of at least one week, at least two weeks, at least three weeks, at least four weeks, at least one month, at least two months or at least three months.

28. A method for treating a hormonal dependent benign or malignant disease of the breast or reproductive tract comprising administering intramuscularly to a subject in need the intramuscular composition comprising compound I, 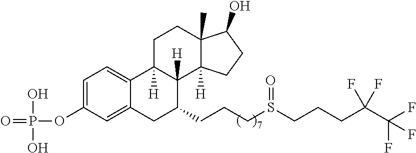
pharmaceutically acceptable salts or solvates thereof; and at least one pharmaceutically acceptable excipient, wherein the composition administered to a subject in need thereof has concentration of compound I, pharmaceutically acceptable salts or solvates thereof; from about 10 mg/ml to about 300 mg/ml and maintains blood plasma fulvestrant concentration of about 1 ng/ml to about 500 ng/ml for desired period of time after injecting the composition comprising compound I, pharmaceutically acceptable salts or solvates thereof.

29. The method of treatment according to claim 28, wherein the desired period of time is selected from group consisting of at least one week, at least two weeks, at least three weeks, at least four weeks, at least one month, at least two months or at least three months.

30. The composition of claim 1, further comprising:
benzyl benzoate.
